(19) United States
(12) Patent Application Publication
(10) Pub. No.: US 2024/0253002 A1
(43) Pub. Date: Aug. 1, 2024

(54) SURFACTANT-GUIDED SPATIAL ASSEMBLY OF NANOARCHITECTURES

(71) Applicant: National University of Singapore, Singapore (SG)

(72) Inventors: Huilin Shao, Singapore (SG); Zhigang Wang, Singapore (SG)

(73) Assignee: National University of Singapore, Singapore (SG)

(21) Appl. No.: 18/566,554

(22) PCT Filed: Jun. 2, 2022
(86) PCT No.: PCT/SG2022/050377

§ 371 Date: Dec. 1, 2023

(30) Foreign Application Priority Data
Jun. 2, 2021 (SG) 10202105866S

(51) Int. Cl. B01J 13/04; A61K 47/69; B82Y 15/00

(52) U.S. Cl. CPC B01J 13/04; A61K 47/6949; B82Y 15/00

(57) ABSTRACT

Provided herein is a composite metal organic framework encapsulating one or more populations of particles, which particles comprise an outer shell region formed from a surfactant and an inner region comprising one or more of a nanoparticle and/or a plurality of protein molecules. Provided also are methods involving the composite metal organic framework.

### FIELD OF INVENTION

The invention relates to a composite metal organic framework, to a method of making a metal organic framework, and a method of selecting materials for making a metal organic framework, and to uses of the metal organic framework.

### BACKGROUND

The listing or discussion of a prior-published document in this specification should not necessarily be taken as an acknowledgement that the document is part of the state of the art or is common general knowledge.

The assembly of functional nanomaterials into desired architectures has attracted considerable attention. The properties of these architectures depend on not only the characteristics of individual nanoparticle building blocks, but also the spatial geometry of the assemblies. While nanoparticles of different material composition, shape and size can be precisely engineered to achieve diverse functionalities, their controlled assembly and spatial organization remains a challenging task to realize sophisticated architectures.

Several approaches have been developed to improve the integration control. For example, through colloidal self-assembly, nanoparticles modified with distinct, complementary molecules (e.g., proteins and nucleic acids) can organize themselves via various assembly forces (e.g., attractive and repulsive forces). Despite its high specificity, the approach requires dedicated, sequence-specific modifications and becomes increasingly challenging to multiplex. To improve the incorporation versatility, external templates are used to assemble nanoparticles into hybrid architectures. In particular, metal-organic frameworks (MOFs) may have crystalline structures, uniform cavities and tunable properties, making them an attractive matrix to host nanoparticles. Nevertheless, existing preparation approaches lack spatial precision to control nanoparticle distribution and organization within the frameworks.

Therefore, there is a need for an improved metal organic frameworks with highly controlled nanoparticle distribution and organisation, as well as methods for the preparation of such improved metal organic frameworks.

### SUMMARY OF THE INVENTION

The inventors have developed a versatile methodology for preparing composite metal organic frameworks comprising particles formed from a surfactant and a nanoparticle and/or proteins. The structure and properties of the composite metal organic framework may be controlled by tuning the interactions of the surfactant with each of the nanoparticle/protein and an organic linker molecule used to form the metal organic framework.

This methodology utilizes surfactants (e.g. amphiphilic surfactants) to guide nanoparticle arrangement during growth of a metal organic framework (MOF). Exploiting the varied interactions of surfactants (polar heads and hydrophobic tails) with MOF constituents and nanoparticles, respectively, the invention allows for precise spatial control of nanoparticle distribution (central vs. peripheral) and organization (clustered vs. dispersed). Through rational selection of surfactants, nanoparticles (and combinations) can be precisely integrated and positioned within various hosts to form different products (e.g., 1D oriented, 3D epitaxial and amorphous). The preparation is fast and safe (<2 min at room temperature, one-pot synthesis with water as the only solvent), and achieves in situ, templated growth on diverse solid substrates.

The invention therefore provides the following numbered clauses.

1. A composite metal organic framework encapsulating one or more populations of particles, wherein a first population of particles comprise:
an outer shell region formed from a surfactant; and
an inner region comprising one or more of a nanoparticle and/or a plurality of protein molecules.

2. The composite metal organic framework according to Clause 1, comprising a first population of particles and a second population of particles, where the second population of particles comprise:
an outer shell region formed from a surfactant; and
an inner region comprising one or more of a nanoparticle and/or a plurality of protein molecules, and wherein the first population of particles are different to the second population of particles.

3. The composite metal organic framework according to Clause 2, wherein:
the first population of particles are mono-dispersed within the metal organic framework; and
the second population of particles are present as particle aggregates within the metal organic framework.

4. The composite metal organic framework according to Clause 3, wherein:
the interaction energy between the surfactant and the particles and/or protein molecules of the first population of particles is less than −83.7 kJ/mol (−20 kcal/mol), and
the interaction energy between the surfactant and the particles and/or protein molecules of the of the second population of particles is greater than −62.8 kJ/mol (−15 kcal/mol).

5. The composite metal organic framework according to any one of Clauses 2 to 4, wherein:
a majority of said first population of particles are located within a peripheral portion of the metal organic framework; and/or
a majority of said second population of particles are located within a core portion of the metal organic framework.

6. The composite metal organic framework according to Clause 5, wherein:
the interaction energy between the surfactant of the first population of particles and the organic linker material of the metal organic framework is greater than −41.8 kJ/mol (−10 kcal/mol), and
the interaction energy between the surfactant of the second population of particles and the organic linker material of the metal organic framework is less than −50.2 kJ/mol (−12 kcal/mol).

7. The composite metal organic framework according to any one of the previous Clauses, wherein the surfactant of the first population particles, and when present the second population of particles, is independently selected from the group consisting of:
a neutral surfactant (e.g. Tween-20, Tween 40, Tween 80, Triton X-100),
a cationic surfactant (e.g. one or more of the group consisting of cetrimonium bromide, dodecyltrimethylammonium bromide, tetradecyltrimethylammonium bromide (TTAB), and cetyltrimethylammonium chloride), and
an anionic surfactant (e.g. one or more of the group consisting of sodium dodecyl sulfate (SDS), sodium hexadecyl sulfate, sodium tetradecyl sulfate (STS), diethylhexyl sodium sulfosuccinate (AOT), and taurodexycholic acid sodium salt (STDC)).

8. The composite metal organic framework according to Clauses 2 to 7, wherein the weight ratio of the first population of particles to the second population of particles ranges from 1:100 to 100:1, optionally from 1:50 to 50:1, more optionally from 1:20 to 20:1, further optionally from 1:10 to 10:1, further optionally still from 1:5 to 5:1, such as from 1:3 to 3:1, for example from 1:2 to 2:1.

9. The composite metal organic framework according to Clause 1, wherein the first population of particles are located within a peripheral portion of the metal organic framework.

10. The composite metal organic framework according to Clause 9, wherein the interaction energy between the surfactant of the first population of particles and the organic linker material of the metal organic framework is greater than −41.8 kJ/mol (−10 kcal/mol).

11. The composite metal organic framework according to Clause 1, wherein the first population of particles are located within a core portion of the metal organic framework, optionally wherein the composite metal organic framework comprises a second population of particles located within a peripheral portion of the metal organic framework.

12. The composite metal organic framework according to Clause 11, wherein the interaction energy between the surfactant of the first population of particles and the organic linker material of the metal organic framework is less than −50.2 kJ/mol (−12 kcal/mol).

13. The composite metal organic framework according to any one of Clauses 1 or 9 to 12 wherein the first population of particles comprises particles mono-dispersed within the metal organic framework.

14. The composite metal organic framework according to Clause 13, wherein the interaction energy between the surfactant and the nanoparticles and/or protein molecules of the first population of particles is less than −83.7 kJ/mol (−20 kcal/mol).

15. The composite metal organic framework according to any one of Clauses 1 or 9 to 12, wherein the first population of particles comprises particles present as particle aggregates within the metal organic framework.

16. The composite metal organic framework according to Clause 15, wherein the interaction energy between the surfactant and the particles and/or protein molecules of the first population of particles is greater than −62.8 kJ/mol (−15 kcal/mol).

17. The composite metal organic framework according to any one of the preceding Clauses, wherein the first population of particles comprises a first set of quantum dots.

18. The composite metal organic framework according to Clause 17 as dependent on any one of Clauses 2 to 8, wherein the second population of particles comprises a second set of quantum dots, optionally wherein the first and second sets of quantum dots are selected from a red quantum dot, a green quantum dot and a blue quantum dot.

19. The composite metal organic framework according to Clause 18, wherein said first set of quantum dots and second set of quantum dots are configured to emit light having different wavelengths.

20. The composite metal organic framework according to any one of Clauses 1 to 16, wherein the first population of particles comprises nanoparticles selected from one or more of the group consisting of an Au nanoparticle (e.g. Au nanosphere, an Au nanorod), a Fe₃O₄ nanoparticle (e.g. an Fe₃O₄ nanocube, an Fe₃O₄ nanosphere), an Ag nanoparticle (e.g. Ag nanosphere), and a CeO₂ nanoparticle (e.g. CeO₂ nanosphere), optionally wherein the first population of particles comprises nanoparticles selected from one or more of the group consisting of an Au nanoparticle (e.g. Au nanosphere, an Au nanorod), and a Fe₃O₄ nanoparticle (e.g. an Fe₃O₄ nanocube, an Fe₃O₄ nanosphere).

21. The composite metal organic framework according to any one of the preceding Claims, wherein the metal organic framework is a 3-D epitaxial metal organic framework,
optionally wherein the 3-D epitaxial metal organic framework is a zeolitic imidazolate framework, such as ZIF-8 or ZIF-67, for example ZIF-8.

22. The composite metal organic framework according to any one of Clauses 1 to 20, wherein the metal organic framework is a 1-D oriented metal organic framework, optionally wherein the 1-D oriented metal organic framework is selected from one or more of the group consisting of Cu-benzene dicarboxylic acid (Cu-BDC), Cu-benzene tricarboxylic acid (Cu-BTC), Ce-benzene dicarboxylic acid (Ce-BDC), and Ce-benzene tricarboxylic acid (Ce-BTC).

23. The composite metal organic framework according to any one of Clauses 1 to 20, wherein metal organic framework is an amorphous metal organic framework, optionally wherein the amorphous metal organic framework is selected from one or more of the group consisting of Fe-benzene dicarboxylic acid (Fe-BDC) and Fe-benzene tricarboxylic acid (Fe-BTC).

24. The composite metal organic framework according to Clause 1, wherein:
(i) the first population of particles comprises gold nanoparticles, the surfactant of the first population of particles is Tween-20 and the metal organic framework is ZIF-8; or
(ii) the first population of particles comprises gold nanoparticles, the surfactant of the first population of particles is cetrimonium bromide and the metal organic framework is ZIF-8; or
(iii) the first population of particles comprises Fe₃O₄ nanoparticles, the surfactant of the first population of particles is Tween-20 and the metal organic framework is ZIF-8; or
(iv) the first population of particles comprises Fe₃O₄ nanoparticles, the surfactant of the first population of particles is cetrimonium bromide and the metal organic framework is ZIF-8; or
(v) the first population of particles comprises gold nanoparticles, the surfactant of the first population of particles is Tween-20, the composite metal organic framework comprises a second population of particles that comprises Fe₃O₄ nanoparticles, the surfactant of the second population of particles is cetrimonium bromide, and the metal organic framework is ZIF-8; or
(vi) the first population of particles comprises a quantum dot (e.g. a red quantum dot), the surfactant of the first population of particles is Tween-20, and the metal organic framework is Ce-benzene dicarboxylic acid; or
(vii) the first population of particles comprises a first set of quantum dots, the surfactant of the first population of particles is Tween-20, the composite metal organic framework comprises a second population of particles that comprises a second set of quantum dots, the surfactant of the second population of particles is cetrimonium bromide, and the metal organic framework is ZIF-8; or
(viii) the first population of particles comprises CeO₂ nanoparticles, the surfactant of the first population of particles is cetrimonium bromide and the metal organic framework is ZIF-8; or
(ix) the first population of particles comprises Fe₃O₄ nanoparticles, the surfactant of the first population of particles is Tween-20 and the metal organic framework is Cu-benzene tricarboxylic acid; or
(x) the first population of particles comprises Fe₃O₄ nanoparticles, the surfactant of the first population of particles is sodium dodecyl sulfate and the metal organic framework is ZIF-67.

25. A method of making a composite metal organic framework encapsulating a first population of particles, said method comprising the steps:
(a) providing a first population of particles, a metal node material and an organic linker material;
(b) reacting said first population of particles, metal node material and organic linker material in aqueous solution to form a composite metal organic framework where the first population of particles are entrapped by the metal organic framework,
   wherein said metal node material and organic linker material are compatible to react together to form a metal organic framework, and
   the first population of particles comprise:
      an outer shell region formed from a surfactant; and
      an inner region comprising one or more of a nanoparticle and/or a plurality of protein molecules.

26. The method according to Clause 25, wherein step (b) is performed on or in the presence of a solid substrate (e.g. polystyrene beads, cellulose fibre, copper wire or a glass slide).

27. The method according to Clause 25 or 26, wherein:
(i) the first population of particles, and
(ii) the metal organic framework formed by said metal node material and organic linker material,
   are as defined in any one of Clauses 9 to 17 or 20 to 23.

28. The method according to Clause 25 or 26, further comprising providing a second population of particles in step (a), and reacting the second population of particles with the first population of particles, metal node material and organic linker material in aqueous solution in step (b), wherein
the second population of particles comprise:
an outer shell region formed from a surfactant; and
an inner region comprising one or more of a nanoparticle and/or a plurality of protein molecules.

29. The method according to Clause 28, wherein:
(i) the first population of particles,
(ii) the second population of particles, and
(iii) the metal organic framework formed by said metal node material and organic linker material,
   are as defined in any one of Clauses 3 to 8, 18 or 19.

30. The method according to any one of Clauses 25, 26 or 28, wherein the first population of particles comprises nanoparticles selected from one or more of the group consisting of an Au nanoparticle (e.g. Au nanosphere, an Au nanorod), a Fe₃O₄ nanoparticle (e.g. an Fe₃O₄ nanocube, an Fe₃O₄ nanosphere), an Ag nanoparticle (e.g. Ag nanosphere), and a CeO₂ nanoparticle (e.g. CeO₂ nanosphere), optionally wherein the first population of particles comprises nanoparticles selected from one or more of the group consisting of an Au nanoparticle (e.g. Au nanosphere, an Au nanorod), and a Fe₃O₄ nanoparticle (e.g. an Fe₃O₄ nanocube, an Fe₃O₄ nanosphere).

31. The method according to any one of Clauses 25 to 30, wherein:
(a) said metal node material is selected from one or more of the group consisting of Zn²⁺, Co²⁺, Cu²⁺, Fe³⁺, and Ce³⁺, optionally one or more of the group consisting of Zn(NO₃)₂, CoCl₂, CuSO₄, FeCl₃, and Ce(NO₃)₃; and/or
(b) said organic linker material is selected from one or more of the group consisting of 2-methylimidazole (HMIM), benzene-1,4-dicarboxylic acid (BDC) and benzene-1,3,5-tricarboxylic acid (BTC).

32. The method according to Clause 25 or 26, wherein the first population of particles, metal node material, organic linker material, and when present the second population of particles, are selected to provide a composite metal organic framework as defined in Clause 24.

33. The method according to any one of Clauses 25 to 32, wherein when the first, and when present second, populations of particles comprises a nanoparticle, the method comprises preparing the first, and when present second, population of particles by:
(i) dispersing said nanoparticles in an organic solvent;
(ii) adding an aqueous solution of said surfactant and mixing the organic and aqueous surfactant solutions; and
(iii) evaporating the organic solvent.

34. The method according to any one of Clauses 25 to 33, further comprising the preliminary steps:
(1) determining the desired distribution and dispersion of each population of particles within the resulting composite metal organic framework; and
(2) for each population of particles, selecting the surfactant, the nanoparticle or protein molecule, the metal node material and the organic linker material compatible to form the desired metal organic framework such that:
   (A) if a majority of said population of particles is desired to exist as mono-dispersed particles, selecting the surfactant and the particle and/or protein molecule such that the interaction energy between the surfactant and the particles and/or protein molecules of said population of particles is less than −83.7 kJ/mol (−20 kcal/mol),
   (B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is greater than −62.8 kJ/mol (−15 kcal/mol),
   (C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is greater than −41.8 kJ/mol (−10 kcal/mol), and
   (D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is less than −50.2 kJ/mol (−12 kcal/mol).

35. A method of selecting materials for forming a composite metal organic framework,
said composite metal organic framework encapsulating one or more populations of particles, where each population of particles comprises:
   an outer shell region formed from a surfactant; and
   an inner region comprising one or more of a nanoparticle and/or a plurality of protein molecules,
said method comprising:
(1) determining the desired distribution and dispersion of each population of particles within the resulting composite metal organic framework; and
(2) for each population of particles, selecting the surfactant, the nanoparticle or protein molecule, and a metal node material and organic linker material compatible to form the desired metal organic framework such that:
(A) if a majority of said population of particles is desired to exist as mono-dispersed particles, selecting the surfactant and the particle and/or protein molecule such that the interaction energy between the surfactant and the particles and/or protein molecules of said population of particles is less than −83.7 kJ/mol (−20 kcal/mol),
(B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is greater than −62.8 kJ/mol (−15 kcal/mol),
(C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is greater than −41.8 kJ/mol (−10 kcal/mol), and
(D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is less than −50.2 kJ/mol (−12 kcal/mol).

36. Use of a composite metal organic framework as defined in any one of Clauses 1 to 24:
(a) as a probe; or
(b) as a catalyst; or
(c) in encryption; or
(d) in fingerprint detection; or
(e) in a therapeutic delivery system.

37. A composite metal organic framework as defined in Clause 18 or 19 for use in a method of diagnosis, wherein said method of diagnosis comprises:
(a) contacting the composite metal organic framework with a biological sample;
(b) measuring the photoluminescence of the first and second set of quantum dots;
(c) determining the presence or absence of a biological marker indicated in a disease state by comparing the photoluminescence of the first and second set of quantum dots with each other or with a reference value, where said biological marker, if present, is able to selectively quench fluorescence of the first or second set of quantum dots; and
(d) assigning the presence or absence of a disease state based on the presence or absence of said biological marker.

38. A diagnostic method comprising:
(a) contacting a composite metal organic framework as defined in Clause 18 or 19 with an isolated biological sample;
(b) measuring the photoluminescence of the first and second set of quantum dots;
(c) determining the presence or absence of a biological marker indicated in a disease state by comparing the photoluminescence of the first and second set of quantum dots with each other or with a reference value, where said biological marker, if present, is able to selectively quench fluorescence of the first or second set of quantum dots; and
(d) assigning the presence or absence of a disease state based on the presence or absence of said biological marker.

39. A composite metal organic framework according to any one of Clauses 1 to 16 and 21 to 23, wherein the nanoparticle is doxorubicin and/or the protein molecule is bovine serum albumin (BSA).

40. A composite product comprising:
a substrate; and
a composite metal organic framework according to any one of Clauses 1 to 24, or 39, wherein the composite metal organic framework is coated over the whole or part of the surface of said substrate.

41. A composite product according to Clause 40, wherein the substrate is selected from the group consisting of polystyrene beads, cellulose fibre, copper wire and a glass slide.

42. A pharmaceutical composition comprising a composite metal organic framework according to Clause 1, wherein the inner region of the first population of particles comprises:
a nanoparticle comprising a small molecule active agent; and/or
a plurality of protein molecules.

The composite metal organic framework of the invention may also be referred to herein as “STAR” (surfactant tunable spatial architecture).

### BRIEF DESCRIPTION OF THE FIGURES

FIG. 1 depicts the surfactant-guided spatial assembly of nano-architectures. a) Schematics of the surfactant-guided nanoparticle integration. The integration performance is determined by surfactant interactions with MOF constituents and nanoparticles. Specifically, strong interaction between surfactants (polar heads) and MOF constituents drives central incorporation and heterogeneous MOF growth around the incorporated nanoparticles, while weak interaction induces tangential integration and homogeneous MOF growth. Likewise, strong interaction between surfactants (hydrophobic tails) and nanoparticles stabilize nanoparticle dispersion while weak interaction leads to nanoparticle aggregation. Through surfactant matching to tune these interactions, the spatial distribution and organization of nanoparticles within MOF hosts can be precisely controlled. The approach is universal for diverse nanoparticles and MOFs, and enables rapid, aqueous synthesis (in solution and in situ on various substrates); b) Schematics and transmission electron micrographs (TEM) of surfactant tunable spatial architecture (STAR, Au-ZIF-8) assemblies. Left: Tween 20-guided assembly. Nanoparticles are peripherally dispersed with respect to the MOF host. Right: CTAB-guided assembly. Aggregated nanoparticles are centrally encapsulated within the MOF host. Scale bars, 50 nm; c) Dual-probe STAR assay for the direct profiling of extracellular vesicle (EV) glycans. In the presence of target glycans, the specific binding of lectin-oxidase mediates in situ generation of hydrogen peroxide (H₂O₂), which selectively quenches the fluorescence of the peripheral working probes, while leaving the central reference probes in the same assemblies unaffected; and d) Microfluidic device and smartphone-based optical detection platform. The microfluidic device consists of two channels: a lower channel with in situ grown STARs and an upper channel with preloaded lectins. Solution mixing between the two channels during assay workflow enables specific targeting and signal generation.

FIG. 2 depicts the characterization of the different MOFs. a) Schematic of metal nodes and organic linkers; b) Zeta potential of MOFs measured in water; c) Photographs (insert) of aqueous MOF suspensions with different optical properties, and TEM images of MOFs displaying various morphologies, including 3D epitaxial zeolitic imidazolate framework (ZIF-8) and ZIF-67, 1D-oriented Cu-BDC, Cu-BTC, Ce-BDC and Ce-BTC, and amorphous Fe-BDC and Fe-BTC; and d) Powder X-ray diffraction (PXRD) analysis confirmed the high crystallinity of the 3D epitaxial MOFs (ZIF-8, ZIF-67) and 1D-oriented MOFs (Cu-BDC, Cu-BTC, Ce-BDC, Ce-BTC) and the weak crystallinity of the amorphous products (Fe-BDC, Fe-BTC). All measurements were performed in triplicate and the data are displayed as mean±SD in c. All unlabeled scale bars, 200 nm.

FIG. 3 depicts the characterization of the different nanoparticles. a) TEM images of nanoparticles, including gold nanosphere and nanorod (Au NS and Au NR), silver nanosphere (Ag NS), magnetic nanocube and nanosphere (Fe₃O₄ NC and Fe₃O₄ NS), cerium dioxide nanosphere (CeO₂ NS); and b) Quantum dots (QDs) emitting blue, green and red fluorescence respectively (BQD, GQD and RQD). Emission spectra of the QDs were measured under UV excitation (365 nm). Scale bars, 20 nm.

FIG. 4 depicts the surfactant effects on STAR assembly. a) Schematic of surfactant interactions with MOFs and nanoparticles. The surfactant's hydrophilic head interacts with MOF constituents (e.g., ZIF-8) and its hydrophobic tail stabilizes nanoparticles (e.g., Au nanospheres); b) Interaction energies between different surfactants and MOF constituents (2-methylimidazole, HMIM), as determined by molecular dynamics simulations; c) Surfactant effects on MOF growth. MOFs were prepared with increasing surfactant doping. MOF size was monitored through dynamic light scattering (DLS) characterization. While sodium dodecyl sulfate (SDS) increased MOF particle size, cetrimonium bromide (CTAB) and Tween 20 suppressed MOF growth; d) Surfactant effects on nanoparticle stability. MOF constituents (Zn²⁺) could exert salt effects on surfactants and affect the stability of surfactant-coated nanoparticles. Tween 20 exhibited the strongest stabilization while surfactants with a charged head (CTAB and SDS) showed extensive nanoparticle aggregation. Nanoparticle aggregation coefficient was determined by d/d₀, where d₀ and d indicate the nanoparticle diameter before and after the addition of Zn²⁺; e) Characterization of STARs prepared with different surfactants. Differentially-coated Au nanospheres were used to prepare various STARs (Au-ZIF-8). Left: TEM images of various assemblies guided by different surfactants. Dashed lines outline MOF structures and white arrows indicate structural distortions. In situ acid wash (after treatment) verified the peripheral dispersion of Tween 20-coated nanoparticles (crosses) and the central encapsulation of CTAB-coated nanoparticles (circle). Right: corresponding size analyses of MOF host and nanoparticle aggregate as well as quantitative analysis of intra-assembly nanoparticle spatial distribution. Scale bars, 50 nm; and f) Effects of nanoparticle loading concentration. By tuning the loading of Au nanospheres, different-sized STARs with consistent nanoparticle organization and distribution could be obtained. n=5 μg. All measurements were performed in triplicate and the data are displayed as mean±SD in b-c, e-f, and as mean in d.

FIG. 5 depicts the molecular dynamics simulation. a) Molecular structures of the surfactant heads and ZIF-8 constituents; and b) Top: potential energies computed from molecular dynamics simulations. Bottom: simulation snapshots. For ionic surfactants, interactions were studied with and without counter ions. All simulations were set with the following parameters: temperature at 25° C., duration of 50 ps with a time step of 1 fs. Simulation data collected in the last 40 ps were used for structural and statistical analysis.

FIG. 6 depicts the surfactant effects on ZIF-8 growth. a) Surfactant effects on MOF diameter. ZIF-8 particles were grown in the presence of various surfactants and the particle diameter was determined through DLS analysis in real time; b) Photographs of MOF suspensions prepared at four surfactant concentrations. For Tween 20 and CTAB samples, the opacity of the suspensions decreased with increasing surfactant loading, consistent with decreased MOF production in the suspensions; and c) TEM images of MOF products prepared with respective surfactants (0.5 mM). All measurements were performed in triplicate and the data are displayed as mean±SD in a.

FIG. 7 depicts the salt-induced aggregation of Au nanospheres. a) Diameter distribution (upper) and UV-Vis absorbance (lower) of Au nanospheres before and after the addition of Zn²⁺. The increased diameter and red-shift in absorbance peak verified the extent of Au nanosphere aggregation (SDS>CTAB>Tween 20); b) Average diameter of Au nanospheres under different conditions. Free surfactants could inhibit the aggregation. The resuspension in H₂O could not reverse the Au aggregation. Au nanospheres were used at 1 mg/ml, in the presence of 0.05 M of Zn²⁺; c) Schematic of Au nanosphere aggregation induced by MOF constituents. Salt can decrease the critical micelle concentration of surfactants by decreasing the electrostatic repulsion between the surfactant head groups (Corrin, M. L. & Harkins, W. D., J. Am. Chem. Soc. 1947, 69, 683-688), leading to aggregation of surfactant coated nanoparticles. Surfactants have different susceptibilities to salts (Qazi, M. J. et al., Langmuir 2017, 33, 4260-4268), resulting in distinct aggregation propensities; and d) Surfactant stabilization effects. Au nanospheres were coated with surfactants of identical head groups and different tail lengths (CTAB and DTAB). The longer-tailed surfactant offered a stronger stabilization capability in reducing nanoparticle aggregation. Aggregation coefficient (δ) is determined by dₜ/d₀, where d₀ and dₜ refer to the average diameters of Au nanospheres at the initial and time t, respectively. All measurements were performed in triplicate and the data are displayed as mean±SD.

FIG. 8 depicts the surfactant-dependent nanoparticle integration. a) Left: zeta potential of Au nanospheres, coated with SDS, CTAB and Tween 20, respectively. Right: stepwise photographs of the reaction solutions, demonstrating the formation of STARs; b) Nanoparticle integration into MOF hosts. Left: schematic processes for integrating different surfactant-coated nanoparticles into ZIF-8. Right: tunable nanoparticle distribution and organization in MOF hosts, as determined by the interactions of surfactants with MOF constituents and nanoparticles, respectively; and c) A typical TEM image of Au-ZIF-8 architecture, where the Au nanoparticles were coated with SDS. All measurements were performed in triplicate and the data are displayed as mean±SD in a.

FIG. 9 depicts the in situ acid washes of Au-ZIF-8 architectures. With increasing buffer acidity, peripherally-associated nanoparticles (Tween 20-coated) were dislodged from the MOF host while the centrally-encapsulated nanoparticles (CTAB-coated) remained within the MOF host. Scale bars, 50 nm.

FIG. 10 depicts the Au-ZIF-8 architectures prepared with varied Au loading. Different-sized Au-ZIF-8 architectures were prepared by varying the Au loading (n=5 μg). For Tween 20-coated Au, most of the Au nanospheres were peripherally associated with the MOF host. For CTAB-coated Au, the nanospheres were dominantly encapsulated within the MOF host. Scale bars, 100 nm.

FIG. 11 depicts STAR tunability. a) Different STAR assemblies. Nanoparticles of various shape, size and material were integrated with ZIF-8 through surfactant guiding. TEM characterization and corresponding analysis of nanoparticle spatial distribution confirmed that all architectures formed abide by the predications as determined by surfactant interactions. Scale bars, 50 nm; b) Tunable nanoparticle spatial distribution. Through varying the loading ratio of nanoparticle populations (i.e., Au nanospheres coated with Tween 20 and CTAB), precise control of intra-assembly nanoparticle distribution was achieved. The STAR morphology correlated well with the initial nanoparticle loading ratio; and c) Nanoparticle distribution and organization in multi-particle STARs. Using Au nanospheres and Fe₃O₄ nanocubes, STARs with distinct architectures were prepared through surfactant matching. Top: TEM images confirming the distinct architectures. Au, solid arrow; Fe₃O₄, dashed arrow. Middle: analysis of nanoparticle distribution with respect to the MOF host. Bottom: analysis of nanoparticle organization with respect to other nanoparticles in the same STAR assemblies. Inter-particle distance was determined through TEM characterization. For each nanoparticle, its average spacing to six nearest Au (x-axis) and Fe₃O₄ (y-axis) particles in the same STAR assembly was plotted. Scale bars, 100 nm. All measurements were performed in triplicate, and the data are displayed as mean±SD.

FIG. 12 depicts ZIF-8 based STARs. a) Metal node and organic linker for 3D epitaxial ZIF-8 formation; b) Aggregation of surfactant-coated nanoparticles in the presence of Zn²⁺. Nanoparticles were applied at 2 mg/ml; and c) TEM images of the STARs and corresponding analysis of nanoparticle spatial distribution through acid buffer wash (HCl buffer, pH=4). The approach demonstrated good universality in tuning the spatial integration of diverse nanoparticles in ZIF-8. NS: nanosphere; NC: nanocube; NP: nanopyramid; NR: nanorod. All measurements were performed in triplicate and the data are displayed as mean±SD. Scale bars, 50 nm.

FIG. 13 depicts ZIF-67 based STARs. a) Metal node and organic linker for 3D epitaxial ZIF-67 formation; b) Diameter of ZIF-67 particles in the presence of free surfactants (0.5 mM). A similar surfactant effect was observed on ZIF-67 growth to that on ZIF-8 growth (see FIG. 4c); c) Aggregation of surfactant-coated Au nanospheres in the presence of Co²⁺. Nanoparticles were applied at 2 mg/ml; d) Schematic prediction of nanoparticle integration into ZIF-67. The nanoparticle distribution and organization could be predicated based on the surfactant interactions to nanoparticles and MOF, respectively; and e) TEM images of the STARs and corresponding analysis of nanoparticle spatial distribution through acid buffer wash (HCl buffer, pH=4), consistent with the prediction. NP: nanopyramid; NR: nanorod. All measurements were performed in triplicate and the data are displayed as mean±SD. Scale bars, 50 nm.

FIG. 14 depicts 1D-oriented STARs. a) Schematic of metal nodes and organic linkers for constructing 1D-oriented MOFs; b) Surfactants showed weak interactions to the 1D-oriented MOFs, as verified by minimal changes to the MOF diameter in the presence of free surfactants (1 mM) using Cu-BDC as a model; c) MOF constituents (e.g., Ce³⁺, Cu²⁺) induced nanoparticle aggregation. Nanoparticle aggregation was surfactant-dependent (aggregation coefficient: SDS>CTAB>Tween 20); d) Schematic prediction of nanoparticle integration in 1D-oriented MOFs. As predicated, the weak interaction of surfactants to 1D-oriented MOFs induces peripheral nanoparticle association with the MOF hosts. Nanoparticle aggregation is surfactant-dependent; and e) TEM images of the STARs and corresponding analysis of nanoparticle spatial distribution through buffer wash. The nanoparticles were dominantly outside the MOF hosts, dispersed (Tween 20) or aggregated (CTAB, SDS), which agreed well with the prediction. HCl buffer (pH=3) and NaOH buffer (pH=9) were used for the analysis of BTC-based and BDC-based STARs, respectively. NS: nanosphere; NP: nanopyramid; NC: nanocube. All measurements were performed in triplicate and the data are displayed as mean±SD. Scale bars, 50 nm.

FIG. 15 depicts amorphous STARs. a) Schematic of metal nodes and organic linkers for constructing amorphous Fe-BDC and Fe-BTC; b) Surfactants showed weak interactions to the hosts, as verified by minimal changes to the formed diameter in the presence of free surfactants (1 mM) using Fe-BTC as a model; c) Constituents (e.g., Fe³⁺) induced nanoparticle aggregation. Nanoparticle aggregation was surfactant-dependent; d) Schematic prediction of nanoparticle integration in amorphous products. As predicated, the weak interaction of surfactants to hosts induces peripheral nanoparticle association. Nanoparticle aggregation is surfactant-dependent; and e) TEM images of the STARs and corresponding analysis of nanoparticle spatial distribution through buffer wash. Most nanoparticles are found outside the hosts, dispersed (Tween 20) or aggregated (CTAB, SDS), which agreed with the prediction. HCl buffer (pH=2) and NaOH buffer (pH=9.5) were used for the analysis of BTC-based and BDC-based STARs, respectively. NS: nanosphere; NC: nanocube. All measurements were performed in triplicate and the data are displayed as mean±SD. Scale bars, 50 nm.

FIG. 16 depicts the nanoparticle spatial distribution and organization in MOFs. a) Single-nanoparticle system. Au-ZIF-8 assemblies were prepared by regulating the loading ratio of Au-Tween 20 and Au-CTAB. The prepared assemblies showed controlled nanoparticle distribution and organization. Specifically, when more Tween 20-coated nanoparticles were loaded, more nanoparticles resided as mono-dispersed outside the MOFs; when more CTAB-coated nanoparticles were used, more nanoparticle aggregates were observed encapsulated within the MOFs. Scale bars, 50 nm; and b) Multi-nanoparticle system. By surfactant matching, Au nanospheres and Fe₃O₄ nanocubes were variedly integrated into the MOF system. TEM analysis verified the nanoparticle spatial distribution and organization within the MOF structure. The nanoparticle aggregation status was characterized by measuring the distance between nanoparticles (interparticle distance). For instance, the three solid arrows mark the distances from one Au nanosphere to three neighboring Fe₃O₄ nanocubes and the three dashed arrows mark the distances from one Fe₃O₄ nanocube to three neighboring Au nanospheres. Scale bars, 100 nm.

FIG. 17 depicts the rapid STAR assembly for biotechnology applications. a) Rapid assembly of STARs. Using different nanoparticles and MOF hosts, STARs were prepared as a suspension through aqueous synthesis. The reaction was completed in <2 min at room temperature and the formed assemblies presented different properties. The nanoparticle integration efficiency (IE) was determined by measuring the amount of residual nanoparticles in the supernatant; b) In situ growth of STARs on diverse substrates. Polystyrene beads: scanning electron micrograph (SEM) (top) and fluorescence microscopy image (bottom) of RQD-ZIF-8 grown on beads. Cellulose fiber: SEM (top) and fluorescence microscopy image (bottom) of RQDZIF-8 grown on fibers. Copper wire: pseudo-colored SEM image of copper wire carrying Fe₃O₄-ZIF-67 (top) and photograph demonstrating its magnetic response (bottom). Templated growth: bright-field (top) and fluorescence (bottom) images of the selective growth of GQD-Ce-BDC; the growth aligned to the seeding pattern as outlined by triglycerides; c) STARs as nanocatalysts. Using differentially-coated Au nanoparticles and ZIF-8 as a host, we prepared STARs with distinct spatial distributions of nanoparticles. The dispersed Au-ZIF-8 demonstrated a much higher catalytic efficiency than the encapsulated version in transforming 4-nitrophenol (4-NP) to 4-aminophenol (4-AP); d) STARs for data encryption. STARs were prepared as combinations of MOF hosts and surfactant-coated nanoparticles. Due to their material composition, the STARs exhibit different optical properties (e.g., color under ambient lighting and fluorescence under UV excitation); due to their intra-assembly spatial distribution of nanoparticles, the STARs respond differently to external stimuli (e.g., acetic acid) to reveal the encrypted code; and e) Fingerprint detection. Bright-field and fluorescence images of the formed RQD-Ce-BDC along the fingerprint lines. All measurements were performed in triplicate, and the data are displayed as mean±SD in a and c.

FIG. 18 depicts the nanoparticle integration efficiency. a) Standard curves were measured to determine nanoparticle concentrations: UV-Vis absorbance at 524 nm for Au nanosphere, UV-Vis absorbance at 480 nm for Fe₃O₄ nanocube, and fluorescence at 519 nm for GQD; and b) Controlled nanoparticle loading into STARs. By increasing the initial nanoparticle concentration in the reaction solution, more nanoparticles were loaded into STARs. All measurements were performed in triplicate and the data are displayed as mean±SD.

FIG. 19 depicts the effects of MOF morphology. a) Fluorescence spectra of STAR composites. QDs were incorporated into 1D-oriented Ce-BDC (top) or 3D epitaxial ZIF-8 (bottom). Both products were quantified to contain an equal amount of respective QDs. The products showed a similar fluorescence profile, indicating the negligible influence of MOF host morphology on the fluorescence properties of the STAR composites; and b) Cellular toxicity of STAR composites. Different STAR composites were incubated with epithelial cells (A431) for 24 hours. Cellular toxicity was evaluated through the MTS proliferation assay. The choice of MOF hosts (more so than the choice of nanoparticles) exerted a strong effect on cellular toxicity. All measurements were performed in triplicate and the data are displayed as mean±SD.

FIG. 20 depicts STAR incorporation of biomolecules. a) Schematic of (RQD-Tween 20)-ZIF-8 with co-integrated therapeutic drug doxorubicin (DOX) and protein bovine serum albumin (BSA); b) Typical TEM image of the composite; c) Loading amount of BSA and DOX; d) Photographs of the developed composite in water. The composite was red under ambient light due to the loaded DOX and demonstrated fluorescence properties, consistent with its RQD constituent; and e) UV-Vis absorption spectra of DOX and the corresponding standard curve (measured at 482 nm) for DOX quantification. All measurements were performed in triplicate and the data are displayed as mean±SD.

FIG. 21 depicts STAR growth on different substrates. a) SEM images of pristine polystyrene bead and cellulose fiber; and b) Templated growth of GQD-Ce-BDC along a seeding pattern made of triglycerides on a glass slide.

FIG. 22 depicts STAR as a nanocatalyst. Two types of Au-ZIF-8 with different spatial distribution of Au nanospheres, dispersed vs. encapsulated, were prepared as catalysts for the transformation of 4-NP to 4-AP in the presence of reductive NaBH₄. The dispersed version was prepared through Au-Tween 20 and the encapsulated through Au-CTAB. By absorbance measurement, we demonstrated that the dispersed STAR has a higher catalytic efficiency than the encapsulated version. The pure ZIF-8 control showed negligible catalytic activity.

FIG. 23 depicts the encryption array. a) Typical STARs with unique optical features (color and fluorescence) for the development of the encryption chip. STARs were embedded within a polyacrylamide gel and patterned on a poly(methyl methacrylate) (PMMA) array; b) Stimulus-induced response. (GQD-Tween 20, Au-SDS)-ZIF-67 was used. It appeared dark violet under ambient light and showed green fluorescence under UV excitation. Upon stimulus treatment (2% acetic acid), ZIF-67 was destroyed and turned colorless. Consequently, the aggregated Au-SDS became visible and appeared grey under ambient light. The structure continued to emit green fluorescence as the GQD remained intact after the stimulus treatment; and c) Different STARs used for information encryption. The assemblies were made of different combinations of nanoparticles and MOFs, and demonstrated different optical responses (color and fluorescence) upon stimulus.

FIG. 24 depicts the smartphone-based STAR microfluidic assay. a) Exploded schematic of the STAR assay cassette 100. The cassette comprises an upper microfluidic channel 200 including an inlet 1 201, an inlet 2 202, an alignment cross 203, preloaded lectins 300 in the lectin chambers 204, and an outlet 205, and a lower microfluidic channel 400 including an inlet 1 401, detection chambers 402, and with dual probe STAR in the detection chambers 403. The two layers (200 and 400) are interconnected though the connection channels 403; b) Operation of the microfluidic platform. (i) Step 1: Analyte solution was introduced into the detection channels through inlet 201, allowing vesicles to be captured onto the STARs. The chambers were blocked subsequently (BSA, 2% w/w); (ii) Step 2: Streptavidin-conjugated enzyme (glucose oxidase, GOD) solution was introduced into the lectin chambers 204 through inlet 202. The preloaded lectins 300 were dissolved and formed lectin-enzyme complexes through streptavidin-biotin interaction. The mixture was then introduced into the detection chambers 402 to allow the specific binding of lectins with corresponding glycan moieties of the captured vesicles; and (iii) Step 3: Glucose solution was introduced into the detection chambers to generate H₂O₂ through GOD-catalyzed reaction. H₂O₂ could position-selectively quench the probe fluorescence of STARs. All fluorescence signals were measured through a smartphone-based optical platform; and c) Schematic of the smartphone-based detection system. The smartphone-based detection system 500 includes a smartphone camera 501, lens 502, filters 503, a chip 504, a diffuser 505, and an UV LED 506. Different fluorescence measurements could be performed through varying the filter configuration.

FIG. 25 depicts the glycan profiling of EVs. a) Schematic of the STAR glycan assay. A dual-probe STAR architecture was developed on a microfluidic platform. It contained GQDs (right dashed arrow) peripherally dispersed and RQDs (left solid arrow) centrally encapsulated within the MOF as the working probes and reference probes, respectively; b) Fluorescence changes. In the presence of target glycan, the specific binding of lectin-oxidase mediates in situ generation of H₂O₂, which selectively quenches the fluorescence of the peripheral working probes, while leaving the central reference probes in the same STARs unaffected. Typical fluorescence spectra showed intensity changes (I_{G} and I_{R}) before and after the assay; c) Robust performance in the presence of interfering agent. Only the STAR assembly accurately revealed the target glycan concentration, amidst the interfering agent (Fe³⁺, 1 μM); d) Detection sensitivity of the assay. The limit of detection was assessed by titrating a known quantity of the target glycoprotein (transferrin) and measuring the associated signal response. The detection limit for ELISA was independently assessed based on fluorescence signals; e) Correlation between STAR and ELISA measurements, when measured with six lectins; f) Glycan profiling in EVs. Left: comparison of glycan profiles between EVs and their parent cells. ΔResponse=Response_{EV}−Response_{Cell}. Right: comparison of glycan profiles between EVs derived from different cell origins. All EV measurements were performed with an equal vesicle concentration (5×10⁸/mL) through the multiplexed STAR platform. Datasets were normalized to their respective highest. NS, not significant; *p<0.05, **p<0.01, ***p<0.001 by two-tailed Student's t-test; g) STAR analysis of 25 lectin markers in clinical cancer ascites (n=12). Hierarchical clustering of patient profiling data classified the patients into two populations and the glycan expressions into two clusters; and h) Principal component analysis of Cluster 1 glycans for prognosis classification. Ellipses were drawn at 95% confidence. All measurements were performed in triplicate and the data are displayed as mean±SD in d-f, and as mean in g-h. a. u., arbitrary units.

FIG. 26 depicts the STAR for enhanced detection of glycans. a) Minimal leaching of QD probes in STAR. To evaluate the extent of nanoparticle leaching from MOF, we prepared a dual-probe STAR (RQD inside and GQD outside). We incubated the structure in the assay buffer, and periodically extracted the composite to monitor its fluorescence intensities over time; b) Time-resolved fluorescence spectra of the dual-probe STAR in the absence (control) and presence of a model target (transferrin). The response was determined by R=1−(Sₜ/Sₒ) and S=I_{G}/I_{R}, where Sₒ is the initial ratiometric fluorescence and Sₜ is the ratiometric fluorescence at time t. I_{G} and I_{R} are the green and red fluorescence intensities, respectively. All measurements were performed in triplicate and the data are displayed as mean±SD; c) The performance of three types of STARs. Different STARs were prepared with QDs (i.e., QD-ZIF-8) and applied to analyze the target in the absence (pure) and presence of an interfering and quenching agent (Fe³⁺, 1 μM); and d) Glycan profiling using 25 lectins on two model glycoproteins, transferrin and ovalbumin. Measurements were performed through both STAR and ELISA assay. All measurements were made with an equal concentration of protein component, and performed relative to samplematched no-lectin controls. The datasets were normalized individually to the highest signal for each analyte and the mean values are presented as heatmaps.

FIG. 27 depicts EV glycan profiling using the STAR assay. a) EVs isolated from brain glial cells (GLI36) and skin epithelial cells (A431) culture medium. All EVs were characterized with nanoparticle tracking analysis and TEM. Scale bars, 100 nm; and b) Multiplexed glycan profiling using 25 lectins on vesicles derived from brain glial cells (GLI36) and skin epithelial cells (A431). Measurements were performed with an equal vesicle concentration (5×10⁸/ml) across samples through the multiplexed STAR platform. Corresponding cell surface glycans were profiled through ELISA. The datasets were normalized individually to the highest signal for each sample and the mean values are presented as heatmaps.

FIG. 28 depicts the clinical analysis of patient ascites. a) Clinical glycan profiling using 25 lectins on colorectal cancer ascites (n=12 patients). Measurements were performed through the STAR assay. All measurements were made with an equal volume of ascites (5 μl) across samples, and performed relative to sample-matched no-lectin controls. The datasets were normalized globally to the highest signal. Hierarchical clustering analysis of the patient specimens categorized the samples into two distinct groups, showing a good concordance with the independent clinical evaluation of patient prognosis (poor and good prognosis). b) Lectin responses of poor and good prognosis patient groups were compared and analyzed. NS, not significant; *p<0.05, **p<0.01, ***p<0.001 by two-tailed Student's t-test.

### DETAILED DESCRIPTION OF THE INVENTION

The invention provides a composite metal organic framework encapsulating one or more populations of particles, wherein a first population of particles comprise:
an outer shell region formed from a surfactant; and
an inner region comprising one or more of a nanoparticle and/or a plurality of protein molecules.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

The composite metal organic framework of the invention is a material in which a metal organic framework encapsulates one or more populations of particles. In other words, a material in which one or more populations of particles are present within a metal organic framework. Thus, as used herein, the term “encapsulated” or “encapsulating” in the context of the composite metal organic framework encapsulating one or more populations of particles means that the particles are present within the metal organic framework.

Metal organic frameworks are well known to a person skilled in the art, and are a class of compounds comprising metal ions coordinated to organic ligands (also known as organic linkers or organic linker material). Metal organic frameworks are typically (but not always) crystalline materials having a regular array of metal ions coordinated to organic ligands.

A first population of the one or more populations of particles comprises an outer shell region formed from a surfactant; and an inner region comprising one or more of a nanoparticle and/or a plurality of protein molecules.

When used herein, the term “nanoparticle” is intended to refer to particles that have an average hydrodynamic diameter of from 0.1 to 2,000 nm. In more particle embodiments of the invention that may be disclosed herein, the nanoparticles may have an average hydrodynamic diameter of from 1 to 1,000 nm, such as from 100 to 400 nm, such as from 120 to 350 nm.

When a composite metal organic framework comprises a plurality of protein molecules, said protein molecules may be individual or aggregated within the inner region of the first population of particles, and may be suspended in the surfactant or present in a micelle.

Without being bound by theory it is believed that, strong interactions between surfactants (primarily the polar heads) and the organic linker material of a metal organic framework drives heterogeneous metal organic framework growth around the readily-incorporated surfactant molecule, while weak interactions favour homogeneous MOF growth and tangential surfactant integration. On the other hand, strong interactions between surfactants (primarily the hydrophobic tails) and nanoparticles stabilize mono-dispersed nanoparticles while weak interactions induce nanoparticle aggregation. It is therefore believed that by the selection of surfactants to tune and mediate these interactions, it is possible to design and guide nanoparticle integration into metal organic frameworks.

Therefore, without being bound by theory it is believed that the location of the particles within the metal organic framework is dependent on the interaction energy between the surfactant and the organic linker material of the metal organic framework as set out below.
If the interaction between the surfactant and organic linker material of the metal organic framework is weak (e.g. greater than −41.8 kJ/mol (−10 kcal/mol)), then the majority of the particles will be present on the periphery of the metal organic framework.
If the interaction between the surfactant and organic linker material of the metal organic framework is strong (e.g. less than −50.2 kJ/mol (−12 kcal/mol)), then the majority of the particles will be present at the core of the metal organic framework.

Without being bound by theory it is believed that the dispersion state of the particles within the metal organic framework is dependent on the interaction energy between the surfactant and the nanoparticle or protein molecule(s) present at the inner region of the particle as set out below.
If the interaction between the surfactant and nanoparticle or protein molecule is weak (e.g. greater than −62.8 kJ/mol (−15 kcal/mol)), then the particles will be present as aggregates.
If the interaction between the surfactant and nanoparticle or protein molecule is strong (e.g. less than −83.7 kJ/mol (−20 kcal/mol)), then the particles will be present as monodispersed particles.

In some embodiments of the invention that may be mentioned herein, the location and dispersion state of the particles may generally follow the principles above. In other words, a majority of a population of particles may follow the principles above.

In some embodiments an interaction energy mentioned herein may be determined by simulation using the Quench task of Forcite module using the COMPASS force field with a microcanonical (NVE) ensemble with temperature at 25° C., duration of 50 ps and a time step of 1 fs.

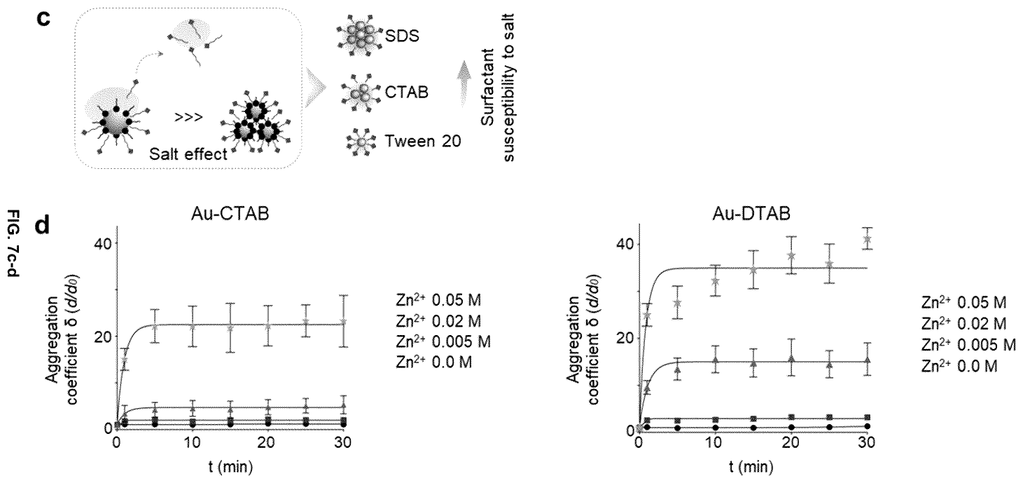

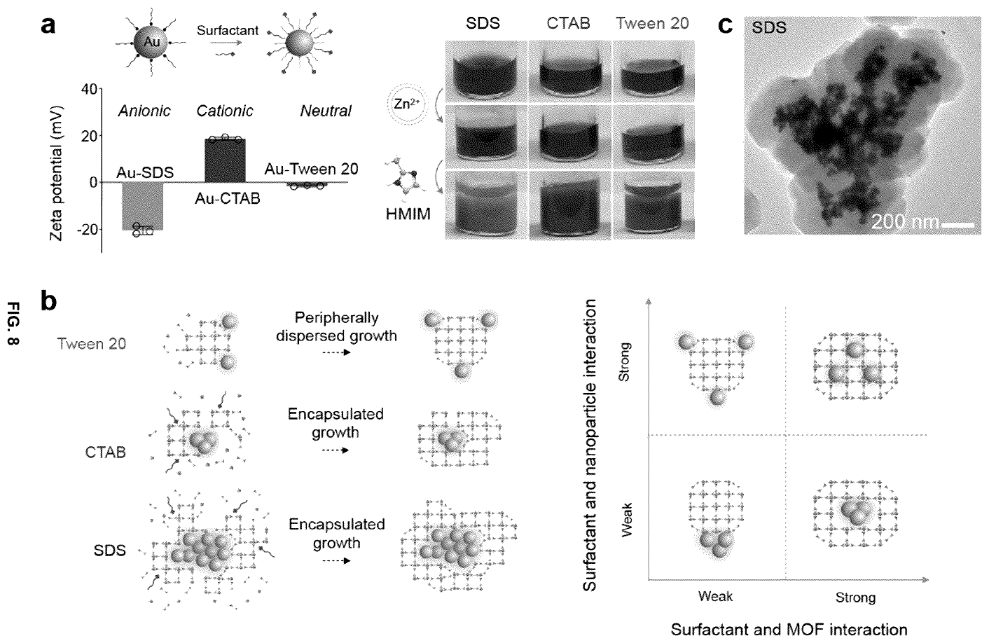

The location and dispersion state of the resulting composite metal organic frameworks is shown graphically in FIG. 8b.

Thus, in some embodiments of the invention that may be mentioned herein, the first population of particles may be located within a peripheral portion of the metal organic framework. In some such embodiments, the interaction energy between the surfactant of the first population of particles and the organic linker material of the metal organic framework may be weak (e.g. greater than −41.8 kJ/mol (−10 kcal/mol)).

In other embodiments of the invention, the first population of particles may be located within a core portion of the metal organic framework. In some such embodiments, the interaction energy between the surfactant of the first population of particles and the organic linker material of the metal organic framework may be strong (e.g. less than −50.2 kJ/mol (−12 kcal/mol)).

In some embodiments of the invention that may be mentioned herein, the first population of particles may comprise particles mono-dispersed within the metal organic framework. In such embodiments, the surfactant and the nanoparticles and/or protein molecules of the first population of particles may be strong (e.g. less than −83.7 kJ/mol (−20 kcal/mol)).

In other embodiments of the invention, the first population of particles may comprise particles present as particle aggregates within the metal organic framework. In such embodiments, the interaction energy between the surfactant and the nanoparticles and/or protein molecules of the first population of particles may be weak (e.g. greater than −62.8 kJ/mol (−15 kcal/mol)).

In some embodiments of the invention that may be mentioned herein, the one or more populations of particles may comprise a first population of particles and a second population of particles, where the second population of particles comprise:
an outer shell region formed from a surfactant; and
an inner region comprising one or more of a nanoparticle and/or a plurality of protein molecules, and
   wherein the first population of particles are different to the second population of particles.

In some such embodiments, one of the populations of particles (e.g. the first population of particles) may be mono-dispersed within the metal organic framework, and the other population of particles (e.g. the second population of particles) may be present as particle aggregates within the metal organic framework. As explained above, this may be achieved by controlling the interactions between the surfactant and the constituent nanoparticle/protein of the first and second population of particles.

In such embodiments, the interaction energy between the surfactant and the particles and/or protein molecules of the mono-dispersed particles (e.g. the first population of particles) may be strong (e.g. less than −83.7 kJ/mol (−20 kcal/mol)), and the interaction energy between the surfactant and the particles and/or protein molecules of the of the particles present as aggregates (e.g. the second population of particles) may be weak (e.g. greater than −62.8 kJ/mol (−15 kcal/mol)).

In some embodiments of the invention that comprise a first population of particles and a second population of particles, a majority of one of the populations of particles (e.g. the first population of particles) may be located within a peripheral portion of the metal organic framework; and a majority of the other population of particles (e.g. the second population of particles) may be located within a core portion of the metal organic framework.

In such embodiments, the interaction energy between the surfactant of the population of particles located within a peripheral portion of the metal organic framework (e.g. the first population of particles) and the organic linker material of the metal organic framework may be weak (e.g. greater than −41.8 kJ/mol (−10 kcal/mol)), and the interaction energy between the surfactant of the population of particles located within a core portion of the metal organic framework (e.g. the second population of particles) and the organic linker material of the metal organic framework may be strong (e.g. less than −50.2 kJ/mol (−12 kcal/mol)).

As used herein, a “majority” means over 50%, for example over 55%, over 60%, over 65%, over 70%, over 75%, over 80%, over 85%, over 90%, over 95%, over 96%, over 97%, over 98%, or over 99%.

For the avoidance of doubt, when the composite metal organic framework comprises two populations of particles, each population may independently involve strong or weak interactions between the surfactant and both of (i) the organic linker material of the metal organic framework, and (iii) the nanoparticle or protein molecule. In other words, the following combinations are explicitly disclosed.

| | First population | | Second population | |
|---|---|---|---|---|
| | Interaction | | Interaction | |
| | between | | between | |
| | surfactant and | | surfactant and | |
| | the organic | | the organic | |
| | linker material | | linker material | |
| | of the metal | | of the metal | |
| | organic | Interaction | organic | Interaction |
| | framework and | between | framework and | between |
| | (iii) the | surfactant and | (iii) the | surfactant and |
| | nanoparticle or | the nanoparticle | nanoparticle or | the nanoparticle |
| | protein | or protein | protein | or protein |
| | molecule | molecule | molecule | molecule |
| 1 | Weak | Weak | Weak | Weak |
| 2 | Weak | Weak | Weak | Strong |
| 3 | Weak | Weak | Strong | Weak |
| 4 | Weak | Strong | Weak | Weak |
| 5 | Strong | Weak | Weak | Weak |
| 6 | Weak | Weak | Strong | Strong |
| 7 | Weak | Strong | Strong | Weak |
| 8 | Weak | Strong | Weak | Strong |
| 9 | Strong | Strong | Weak | Weak |
| 10 | Strong | Weak | Strong | Weak |
| 11 | Strong | Weak | Weak | Strong |
| 12 | Strong | Strong | Strong | Weak |
| 13 | Strong | Strong | Weak | Strong |
| 14 | Strong | Weak | Strong | Strong |
| 15 | Weak | Strong | Strong | Strong |
| 16 | Strong | Strong | Strong | Strong |

It will be appreciated that combinations of more than two different kinds of particles is contemplated (e.g. two, three or four) and that these may be derived by analogy from the table above.

In some embodiments of the invention that may be mentioned herein, the surfactant may be selected from the group consisting of:
a neutral surfactant (e.g. Tween-20, Tween 40, Tween 80, Triton X-100),
a cationic surfactant (e.g. one or more of the group consisting of cetrimonium bromide, dodecyltrimethylammonium bromide, tetradecyltrimethylammonium bromide (TTAB), and cetyltrimethylammonium chloride), and
an anionic surfactant (e.g. one or more of the group consisting of sodium dodecyl sulfate (SODS), sodium hexadecyl sulfate, sodium tetradecyl sulfate (STS), diethylhexyl sodium sulfosuccinate (AOT), and taurodexycholic acid sodium salt (STDC)).

When the composite metal organic framework of the invention comprises two (or more) populations of particles, the surfactant for each population may independently be selected from those listed above. In such cases, a skilled person would understand that since the surfactant influences the location of the particles within the composite metal organic framework, if two populations are desired to be located in different portions of the composite metal organic framework, they will be selected to have different surfactants. Alternatively, if the populations are desired to be located in the same portion of the composite metal organic framework (e.g. both in the core or both in the periphery), the surfactants may be the same. Furthermore, it is herein explicitly contemplated that a second, third, fourth etc population of particles may have any of the properties defined for the first population of particles in the claims below.

In embodiments of the invention comprising more than one population of particles (e.g. a first population of particles and a second population of particles), the populations may be present at any appropriate weight ratio, such as from 1:100 to 100:1. Thus, the weight ratio of a first population of particles to a second population of particles may be from 1:100 to 100:1, for example from 1:50 to 50:1, from 1:20 to 20:1, from 1:10 to 10:1, from 1:5 to 5:1, from 1:3 to 3:1, or from 1:2 to 2:1.

For the avoidance of doubt, it is explicitly contemplated that where a number of numerical ranges related to the same feature are cited herein, that the end points for each range are intended to be combined in any order to provide further contemplated (and implicitly disclosed) ranges.

For example, the following weight ratio ranges are herein explicitly contemplated from the above.

From 1:100 to 100:1, from 1:100 to 50:1, from 1:100 to 20:1, from 1:100 to 10:1, from 1:100 to 5:1, from 1:100 to 3:1, from 1:100 to 2:1, from 1:100 to 1:2, from 1:100 to 1:3, from 1:100 to 1:5, from 1:100 to 1:10, from 1:100 to 1:20, from 1:100 to 1:50;
from 1:50 to 100:1, from 1:50 to 50:1, from 1:50 to 20:1, from 1:50 to 10:1, from 1:50 to 5:1, from 1:50 to 3:1, from 1:50 to 2:1, from 1:50 to 1:2, from 1:50 to 1:3, from 1:50 to 1:5, from 1:50 to 1:10, from 1:50 to 1:20;
from 1:20 to 100:1, from 1:20 to 50:1, from 1:20 to 20:1, from 1:20 to 10:1, from 1:20 to 5:1, from 1:20 to 3:1, from 1:20 to 2:1, from 1:20 to 1:2, from 1:20 to 1:3, from 1:20 to 1:5, from 1:20 to 1:10;
from 1:10 to 100:1, from 1:10 to 50:1, from 1:10 to 20:1, from 1:10 to 10:1, from 1:10 to 5:1, from 1:10 to 3:1, from 1:10 to 2:1, from 1:10 to 1:2, from 1:10 to 1:3, from 1:10 to 1:5;
from 1:5 to 100:1, from 1:5 to 50:1, from 1:5 to 20:1, from 1:5 to 10:1, from 1:5 to 5:1, from 1:5 to 3:1, from 1:5 to 2:1, from 1:5 to 1:2, from 1:5 to 1:3;
from 1:3 to 100:1, from 1:3 to 50:1, from 1:3 to 20:1, from 1:3 to 10:1, from 1:3 to 5:1, from 1:3 to 3:1, from 1:3 to 2:1, from 1:3 to 1:2;
from 1:2 to 100:1, from 1:2 to 50:1, from 1:2 to 20:1, from 1:2 to 10:1, from 1:2 to 5:1, from 1:2 to 3:1, from 1:2 to 2:1;
from 2:1 to 100:1, from 2:1 to 50:1, from 2:1 to 20:1, from 2:1 to 10:1, from 2:1 to 5:1, from 2:1 to 3:1;
from 3:1 to 100:1, from 3:1 to 50:1, from 3:1 to 20:1, from 3:1 to 10:1, from 3:1 to 5:1, from 5:1 to 100:1, from 5:1 to 50:1, from 5:1 to 20:1, from 5:1 to 10:1;
from 10:1 to 100:1, from 10:1 to 50:1, from 10:1 to 20:1;
from 20:1 to 100:1, from 20:1 to 50:1; and
from 50:1 to 100:1.

In some embodiments of the invention that may be mentioned herein, the first population of particles may comprise a first set of quantum dots. This enables the composite metal framework to act as a sensor. The composite metal framework of the invention may be especially advantageous as a probe/sensor when the one or more populations of particles comprise a first population of particles comprising a first set of quantum dots, and a second population of particles comprising a second set of quantum dots. In such cases, the two sets of quantum dots may be configured to emit light having different wavelengths. For example, the two sets of quantum dots may be independently selected from a red quantum dot, a green quantum dot and a blue quantum dot.

It may be particularly advantageous for the composite metal framework to comprise a first set of quantum dots located in a core region of the metal organic framework, and a second set of quantum dots located in a peripheral region of the metal organic framework. This may enable for a different response to various stimuli, depending on whether or not a stimulus is able to penetrate into the core region of the metal organic framework and/or whether a stimulus is able to selectively quench fluorescence of the first or second set of quantum dots. For example, if a stimulus is not able to penetrate into the metal organic framework then it may modulate a signal from a quantum dot located at the periphery of the metal organic framework, while a quantum dot located at the core of the metal organic framework acts as a reference signal. A specific example of such sensing may be the detection of hydrogen peroxide, which may quench the fluorescence of quantum dots. Thus, the metal organic frameworks may be used to detect the presence of biomarkers that are able to generate quenching molecules (e.g. hydrogen peroxide) in the presence of an appropriate substrate (e.g. lectin-oxidase).

In some embodiments of the invention that may be mentioned herein, the first population of particles may comprise nanoparticles selected from one or more of the group consisting of an Au nanoparticle (e.g. Au nanosphere, an Au nanorod), a Fe₃O₄ nanoparticle (e.g. an Fe₃O₄ nanocube, an Fe₃O₄ nanosphere), an Ag nanoparticle (e.g. Ag nanosphere), and a CeO₂ nanoparticle (e.g. CeO₂ nanosphere). In some such embodiments, the first population of particles may comprise nanoparticles selected from one or more of the group consisting of an Au nanoparticle (e.g. Au nanosphere, an Au nanorod), and a Fe₃O₄ nanoparticle (e.g. an Fe₃O₄ nanocube, an Fe₃O₄ nanosphere).

In some embodiments of the invention that may be mentioned herein, the first population of particles may comprise nanoparticles that compromise a pharmaceutical compound, such as a small molecule active agent, e.g. doxorubicin.

In some embodiments of the invention that may be mentioned herein, the first population of particles may comprise a protein, such as bovine serum albumin (BSA).

As will be appreciated by a person skilled in the art, the second (or further) populations of nanoparticles, when present, may be selected from the same options as the first set of nanoparticles.

The metal organic framework may be any suitable metal organic framework, such as a 3-D epitaxial metal organic framework, a 1-D oriented metal organic framework, or an amorphous metal organic framework. Suitable examples of 3-D epitaxial metal organic frameworks include zeolitic imidazolate frameworks (ZIFs), such as ZIF-8 or ZIF-67, for example ZIF-8. Suitable examples of 1-D oriented metal organic frameworks include Cu-benzene dicarboxylic acid (Cu-BDC), Cu-benzene tricarboxylic acid (Cu-BTC), Ce-benzene dicarboxylic acid (Ce-BDC), and Ce-benzene tricarboxylic acid (Ce-BTC). Suitable examples of amorphous metal organic frameworks include Fe-benzene dicarboxylic acid (Fe-BDC) and Fe-benzene tricarboxylic acid (Fe-BTC).

Specific examples of composite metal organic frameworks according to the invention are those in which:
(i) the first population of particles comprises gold nanoparticles, the surfactant of the first population of particles is Tween-20 and the metal organic framework is ZIF-8: or
(ii) the first population of particles comprises gold nanoparticles, the surfactant of the first population of particles is cetrimonium bromide and the metal organic framework is ZIF-8; or
(iii) the first population of particles comprises Fe₃O₄ nanoparticles, the surfactant of the first population of particles is Tween-20 and the metal organic framework is ZIF-8; or
(iv) the first population of particles comprises Fe₃O₄ nanoparticles, the surfactant of the first population of particles is cetrimonium bromide and the metal organic framework is ZIF-8; or
(v) the first population of particles comprises gold nanoparticles, the surfactant of the first population of particles is Tween-20, the composite metal organic framework comprises a second population of particles that comprises Fe₃O₄ nanoparticles, the surfactant of the second population of particles is cetrimonium bromide, and the metal organic framework is ZIF-8; or
(vi) the first population of particles comprises a quantum dot (e.g. a red quantum dot), the surfactant of the first population of particles is Tween-20, and the metal organic framework is Ce-benzene dicarboxylic acid; or
(vii) the first population of particles comprises a first set of quantum dots, the surfactant of the first population of particles is Tween-20, the composite metal organic framework comprises a second population of particles that comprises a second set of quantum dots, the surfactant of the second population of particles is cetrimonium bromide, and the metal organic framework is ZIF-8; or
(viii) the first population of particles comprises CeO₂ nanoparticles, the surfactant of the first population of particles is cetrimonium bromide and the metal organic framework is ZIF-8; or
(ix) the first population of particles comprises Fe₃O₄ nanoparticles, the surfactant of the first population of particles is Tween-20 and the metal organic framework is Cu-benzene tricarboxylic acid; or
(x) the first population of particles comprises Fe₃O₄ nanoparticles, the surfactant of the first population of particles is sodium dodecyl sulfate and the metal organic framework is ZIF-67.

The composite metal organic framework of the invention may be utilised on a substrate. Thus, the invention provides a composite product comprising:
a substrate; and
a composite metal organic framework according to the invention, wherein the composite metal organic framework is coated over the whole or part of the surface of said substrate.

In some embodiments of the invention, the substrate may be selected from the group consisting of polystyrene beads, cellulose fibre, copper wire and a glass slide.

The invention also provides a method of making a composite metal organic framework encapsulating a first population of particles, the method comprising the steps:
(a) providing a first population of particles, a metal node material and an organic linker material;
(b) reacting said first population of particles, metal node material and organic linker material in aqueous solution to form a composite metal organic framework where the first population of particles are entrapped by the metal organic framework,
   wherein said metal node material and organic linker material are compatible to react together to form a metal organic framework, and
   the first population of particles comprise:
      an outer shell region formed from a surfactant; and
      an inner region comprising one or more of a nanoparticle and/or a plurality of protein molecules.

The metal organic framework and first population of particles may have any property defined above.

In some embodiments of the invention that may be mentioned herein, step (b) may be performed on or in the presence of a solid substrate (e.g. polystyrene beads, cellulose fibre, copper wire or a glass slide).

In some embodiments of the invention that may be mentioned herein, the method may involve a second population of particles. Thus, the method may comprise providing a second population of particles in step (a), and reacting the second population of particles with the first population of particles, metal node material and organic linker material in aqueous solution in step (b), wherein
the second population of particles comprise:
   an outer shell region formed from a surfactant; and
   an inner region comprising one or more of a nanoparticle and/or a plurality of protein molecules.

The metal node material may be selected from any appropriate metal node for forming a metal organic framework. Thus, in some embodiments of the invention that may be mentioned herein, the metal node material may be selected from one or more of the group consisting of Zn²⁺, Co²⁺, Cu²⁺, Fe³⁺, and Ce³⁺. Specific compounds that may be useful in the method of the invention include Zn(NO₃)₂, CoCl₂, CuSO₄, FeCl₃, and Ce(NO₃)₃.

The organic linker material may be selected from any appropriate organic linker material for forming a metal organic framework. Thus, in some embodiments of the invention that may be mentioned herein, the organic linker material may be selected from one or more of the group consisting of 2-methylimidazole (HMIM), benzene-1,4-dicarboxylic acid (BDC) and benzene-1,3,5-tricarboxylic acid (BTC).

When the first, and when present second, populations of particles comprises a nanoparticle, the method may comprise preparing the first, and when present second, population of particles by:
(i) dispersing said nanoparticles in an organic solvent;
(ii) adding an aqueous solution of said surfactant and mixing the organic and aqueous surfactant solutions; and
(iii) evaporating the organic solvent.

As discussed herein, the interaction energies between the surfactant and each of the organic linker material of the metal organic framework, and the nanoparticle or protein molecule, are believed to influence the resulting structure of the composite metal organic framework. This allows the species in question to be selected to provide the first, and when present second or further, populations of particles in a desired dispersion state and location of the metal organic framework. Thus, in some embodiments of the invention that may be mentioned herein, the method may comprise the preliminary steps:
(1) determining the desired distribution and dispersion of each population of particles within the resulting composite metal organic framework; and
(2) for each population of particles, selecting the surfactant, the nanoparticle or protein molecule, the metal node material and the organic linker material compatible to form the desired metal organic framework such that:
   (A) if a majority of said population of particles is desired to exist as mono-dispersed particles, selecting the surfactant and the particle and/or protein molecule such that the interaction energy between the surfactant and the particles and/or protein molecules of said population of particles is strong (e.g. less than −83.7 kJ/mol (−20 kcal/mol)),
   (B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is weak (e.g. greater than −62.8 kJ/mol (−15 kcal/mol)),
   (C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is weak (e.g. greater than −41.8 kJ/mol (−10 kcal/mol)), and
   (D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is strong (e.g. less than −50.2 kJ/mol (−12 kcal/mol)).

The invention also provides a method of selecting materials for forming a composite metal organic framework,
said composite metal organic framework encapsulating one or more populations of particles, where each population of particles comprises:
   an outer shell region formed from a surfactant; and
   an inner region comprising one or more of a nanoparticle and/or a plurality of protein molecules,
   said method comprising:
(1) determining the desired distribution and dispersion of each population of particles within the resulting composite metal organic framework; and
(2) for each population of particles, selecting the surfactant, the nanoparticle or protein molecule, and a metal node material and organic linker material compatible to form the desired metal organic framework such that:
   (A) if a majority of said population of particles is desired to exist as mono-dispersed particles, selecting the surfactant and the particle and/or protein molecule such that the interaction energy between the surfactant and the particles and/or protein molecules of said population of particles is strong (e.g. less than −83.7 kJ/mol (−20 kcal/mol)),
   (B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is weak (e.g. greater than −62.8 kJ/mol (−15 kcal/mol)),
   (C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is weak (e.g. greater than −41.8 kJ/mol (−10 kcal/mol)), and
   (D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is strong (e.g. less than −50.2 kJ/mol (−12 kcal/mol)).

In the above methods involving the selection of materials for forming a composite metal organic framework, the interaction energies between the surfactant and other components may be determined by simulation using the Quench task of Forcite module using the COMPASS force field with a microcanonical (NVE) ensemble with temperature at 25° C., duration of 50 ps and a time step of 1 fs.

As discussed in more detail herein, the composite metal organic framework of the invention may be utilised in a number of applications, for example:
(a) as a probe; or
(b) as a catalyst; or
(c) in encryption; or
(d) in fingerprint detection; or
(e) in a therapeutic delivery system.

Suitable components for each of these uses will be apparent to a skilled person from the present disclosure, in particular the below Examples.

The composite metal organic framework may also be useful in a method of diagnosis, particularly when the composite metal organic framework comprises two populations of particles: a first population of particles comprising a first set of quantum dots, and a second population of particles comprising a second set of quantum dots. Such composite metal organic frameworks may be useful in a method of diagnosis, wherein said method of diagnosis comprises:
(a) contacting the composite metal organic framework with a biological sample;
(b) measuring the photoluminescence of the first and second set of quantum dots;
(c) determining the presence or absence of a biological marker indicated in a disease state by comparing the photoluminescence of the first and second set of quantum dots with each other or with a reference value, where said biological marker, if present, is able to selectively quench fluorescence of the first or second set of quantum dots; and
(d) assigning the presence or absence of a disease state based on the presence or absence of said biological marker.

The invention also provides a diagnostic method utilising such metal organic frameworks, for example a diagnostic method comprising:
(a) contacting the composite metal organic framework with an isolated biological sample;
(b) measuring the photoluminescence of the first and second set of quantum dots;
(c) determining the presence or absence of a biological marker indicated in a disease state by comparing the photoluminescence of the first and second set of quantum dots with each other or with a reference value, where said biological marker, if present, is able to selectively quench fluorescence of the first or second set of quantum dots; and
(d) assigning the presence or absence of a disease state based on the presence or absence of said biological marker.

The invention also provides a pharmaceutical composition comprising a composite metal organic framework according to the invention, wherein the inner region of the first population of particles comprises:
a nanoparticle comprising a small molecule active agent; and/or
a plurality of protein molecules.

The invention is illustrated by the below Examples, which are not to be construed as limitative.

### EXAMPLES

In the following Examples, the composite metal organic framework of the invention is also referred to as “STAR” (surfactant tunable spatial architecture).

### Materials

All chemicals used for synthesis and modification were purchased from Sigma-Aldrich and used directly, unless otherwise stated. Dulbecco's modified Eagle's medium (DMEM) and vesicle-depleted fetal bovine serum (dFBS) were purchased from HyClone. Penicillin-streptomycin was purchased from Corning. Polydimethylsiloxane (PDMS) was purchased from Dow Corning. Phosphate buffered saline (PBS) and biotinylated lectins were purchased from Vector Laboratories. Anti-CD24 antibody was purchased from eBioscience of Thermo Fisher Scientific.

### Analytical Techniques

### DLS

DLS analysis of particle diameter and zeta potential was performed with Zetasizer Nano ZS instrument (Malvern).

### PXRD

PXRD was performed in the 26 range 5-50° at a scanning rate of 2° min⁻¹ on an X-ray diffractometer (Bruker D8 Advanced) with a Cu-Kα radiation at 40 KV and 40 mA.

### SEM

For SEM analysis, samples were loaded on silicon slides, sputter-coated with gold (Leica) before being examined (JEOL 6701).

### TEM

For TEM analysis, samples were loaded onto carbon-coated copper grids (Latech) for imaging (JEOL 2010F).

### Statistical Analysis

All measurements were performed in triplicate, and the data are displayed as mean±standard deviation. Correlations were performed with linear regression to determine the goodness of fit (R²). For inter-sample comparisons, multiple pairs of samples were analyzed by two-tailed t-test, and the resulting P values were adjusted for multiple hypothesis testing using Bonferroni correction. For unsupervised hierarchical clustering analysis, STAR profiling of glycan signatures were clustered using Euclidean distance metric and complete linkage (Morpheus, Broad Institute). The lectin markers were grouped into two clusters, according to patient expression profiles. Principal component analysis was performed using Minitab (v.16.1) based on a combination of significant lectin markers to categorize the patient samples according to their clinical prognosis. All other statistical analyses were performed using GraphPad Prism (v. 7.0c).

### Example 1. Preparation of Nanomaterials

### Gold Nanosphere (Au NS)

Au NS was synthesized by rapidly injecting gold precursors into a pre-heated surfactant solution (Peng, S. et al., Proc. Natl. Acad. Sci. USA 2010, 107, 14530-14534). Briefly, oleylamine (5 ml) was refluxed at 150° C. under nitrogen. A mixture of HAuCl₄·3H₂O (0.3 mmol) in oleylamine (1 ml) was rapidly injected into the hot solution and stirred for 1.5 h. The obtained Au NS was washed by ethanol and dispersed in chloroform for further use.

### Gold Nanorod (Au NR)

Au NR was prepared via a seed-mediated growth method (Nikoobakht, B. & El-Sayed, M. A., Chem. Mater. 2003, 15, 1957-1962). Spherical gold seeds were newly synthesized by vigorously mixing ice-cold NaBH₄ (0.01 M, 0.6 ml) and an aqueous solution (7.5 ml) containing CTAB (0.1 M) and HAuCl₄ (0.3 mM). After 5 s of mixing, the seed solution was ready for further use. Au NR were synthesized in a water bath at 29° C. CTAB (5 ml, 0.2 M) was mixed with AgNO₃ solution (4 mM, 0.25 ml), HAuCl₄ (1 mM, 5 ml) and ascorbic acid (70 μl, 78.8 mM). The seed solution (12 μl) was then added to the growth solution and allowed to grow overnight. The obtained Au NR was further purified using CTAB (Jana, N. R., Chem. Commun. 2003, 1950-1951) and stored in water for further use.

Iron Oxide Nanosphere (Fe₃O₄ NS)

Fe₃O₄ NS was synthesized according to a published method (Park, J. et al., Nat. Mater. 2004, 3, 891-895). Fe-oleate complex was first prepared by reacting metal and sodium oleate. The Fe-oleate complex (3.6 g) and oleic acid (1.0 g, 90%) were dissolved in 1-octadecene (10 ml, 90%) at room temperature. The reaction mixture was heated to 320° C. for 30 min under nitrogen atmosphere. The particles were precipitated using ethanol, and dispersed in chloroform.

Fe₃O₄ Nanocube (Fe₃O₄ NC)

Fe₃O₄ NC was prepared through thermal decomposition of Fe-oleate precursors (Muro-Cruces, J. et al., ACS Nano 2019, 13, 7716-7728). In a typical experiment, 1-octadecene (5 ml) solution containing Fe-oleate (2 g) and sodium oleate (2.2 g, 82%) was heated to 320° C. and refluxed for 60 min to allow nanocube growth. The particles were precipitated using ethanol, and dispersed in chloroform.

CeO₂ Nanosphere (CeO₂ NS)

CeO₂ NS was prepared through thermal decomposition of cerium nitrate (Lee, S. S. et al., Chem. Mater. 2012, 24, 424-432). Briefly, Ce(NO₃)₃·6H₂O (0.108 g, 0.25 mmol) and oleylamine (0.802 g, 3.0 mmol) were dissolved in 1-octadecene (4 ml) at 80° C. The resultant mixture was then heated to 260° C. for 2 h. The particles were precipitated using ethanol, and dispersed in chloroform.

### Silver Nanosphere (Ag NS)

Ag NS was prepared by dissolving AgNO₃ (51 mg, 0.30 mmol) in oleylamine (7.5 mmol, 2.5 ml) which was then injected quickly into refluxing toluene (50 ml, Hiramatsu, H. & Osterloh, F. E., Chem. Mater. 2004, 16, 2509-2511). The reaction was left at reflux overnight, before being cooled. The particles were precipitated using ethanol, and dispersed in chloroform.

### Example 2. Preparation of Surfactant-Stabilized Nanoparticles in Aqueous Media

To stabilize hydrophobic nanoparticles in aqueous media, surfactant modification was performed through phase transfer reactions. Briefly, the as-synthesized nanoparticles in Example 1 were dispersed in organic solvent (0.5 ml, 5 mg/ml), and mixed with aqueous surfactant solutions (0.5 ml). The mixture was sonicated for 3 min and organic solvent was evaporated at 80° C. The resultant suspension was filtered to remove excess free surfactants.

### Example 3. Preparation of Quantum Dots Nanopyramide (QD NP)

QD NP were prepared according to reported methods (Chen, D. et al., Chem. Mater. 2010, 22, 1437-1444; and Bae, W. K. et al., Chem. Mater. 2008, 20, 531-539).

For the synthesis of blue QDs (BQD), preliminarily, S precursor solution (0.1 M) was prepared using S powder and 1-octadecene, and Zn precursor solution (0.1 M) was prepared by dissolving ZnO and oleic acid (1:8 molar ratio) in 1-octadecene. To synthesis CdS core, a mixture of CdO (0.2 mmol), oleic acid (1.6 mmol), and 1-octadecene (6 g) was heated to become clear at 260° C. and then S precursor solution (1 ml) was rapidly injected. After 30 min, the reaction mixture with formed CdS nanocrystals was cooled down to 50° C. Then, methanol was used to remove unreacted precursors and side products. To grow the ZnS shell, oleylamine (2 ml) was added to the CdS core solution and heated to 120° C. The Zn and S precursor solutions (1 ml each) were added consecutively. The temperature was increased immediately to 220° C. and kept for 20 min to allow the growth of ZnS shell.

For typical synthesis of green QDs (GQD) and red QDs (RQD), CdO and zinc acetate were dissolved in oleic acid (5 ml) at elevated temperature 150° C. under nitrogen protection. 1-octadecene (10 ml) was then added and the temperature was increased to 310° C. Finally, a stock solution containing trioctylphosphine (3 ml), Se powder and S powder was quickly injected into the reaction. The reaction was maintained at 310° C. for 10 min before cooling to room temperature. The initial precursor compositions corresponding to GQD and RQD were (Cd: 0.4 mmol; Zn: 4 mmol; Se: 0.1 mmol; S: 4 mmol) and (Cd: 0.4 mmol; Zn: 4 mmol; Se: 1 mmol; S: 2.3 mmol), respectively. The product was collected and dispersed in chloroform.

### Example 4. STAR Preparation and STAR Assembly

### STAR Preparation

All reactions were performed in water and at room temperature. Surfactant-stabilized nanoparticles (5 mg/ml, prepared in Example 2), metal nodes including Zn(NO₃)₂ (0.05 M), COCl₂ (0.05 M), CuSO₄ (0.025 M), FeCl₃ (0.025 M) and Ce(NO₃)₃ (0.025 M), as well as organic linkers including HMIM (2.5 M), benzene-1,4-dicarboxylic acid (BDC, 0.025 M) and benzene-1,3,5-tricarboxylate (BTC, 0.025 M), were used for different STAR assemblies, unless otherwise stated. BDC and BTC were dissolved in water, with the addition of NaOH, and the final solution was kept at pH 6-7. Taking (Au-Tween 20)-(ZIF-8) for example, Tween 20-stabilized Au nanoparticles (2.5 μl) were mixed with Zn(NO₃)₂ (50 μl), followed by the addition of HMIM (50 μl). After vigorous mixing, the mixture was allowed to react in static for 10 min. The product was collected through centrifugation and washed by deionized water. All other composites were prepared in a consistent approach.

### Example 5. Characterization of Materials

### Spatial Distribution of Nanoparticles in MOF Host

To examine the spatial distribution of nanoparticles in MOF host, the prepared composites were washed in situ on TEM grid. Specifically, STAR solution (10 μl) was first deposited on a TEM grid for 5 min. Following gentle removal of the suspension, wash buffers of various pH (10 μl) was dropped and incubated on the grid, before being wicked away with a piece of filter paper. The prepared grid was finally dried for further TEM analysis, to evaluate the remaining nanoparticle distribution and morphology in MOF structures, so as to optimize the wash conditions.

### Results and Discussion

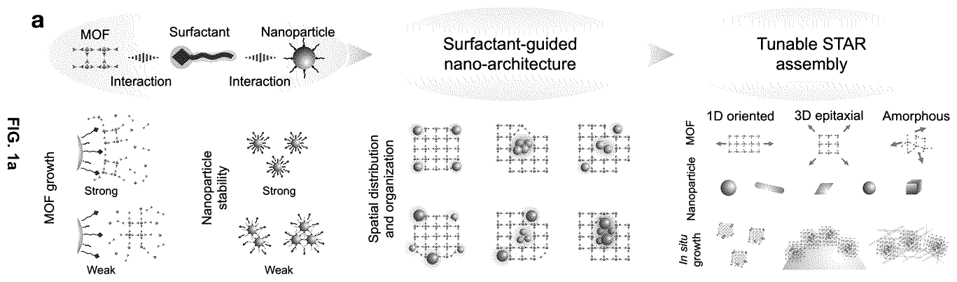

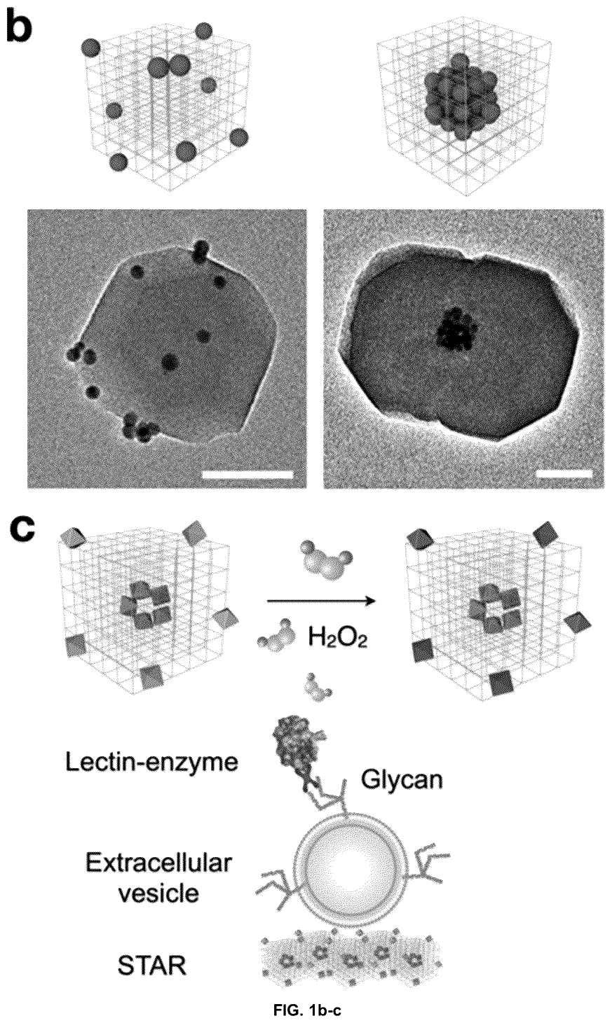

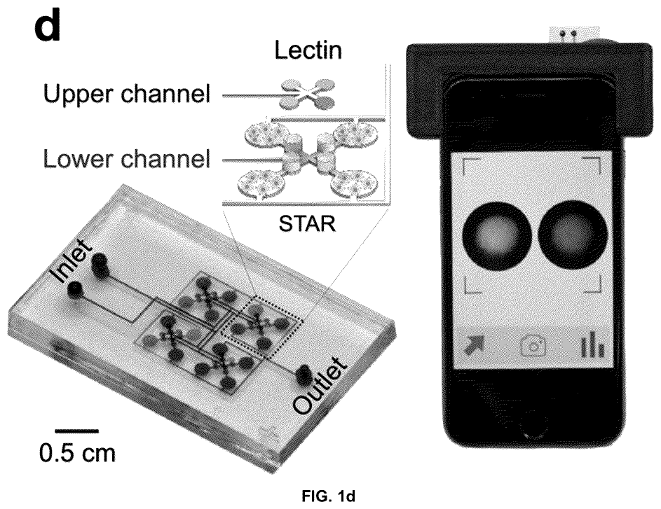

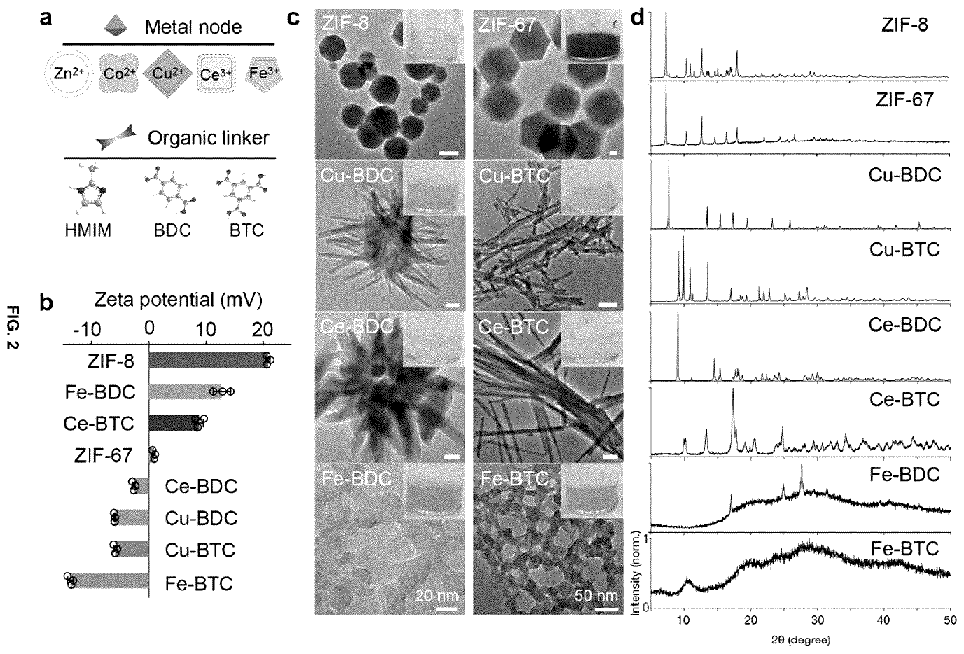

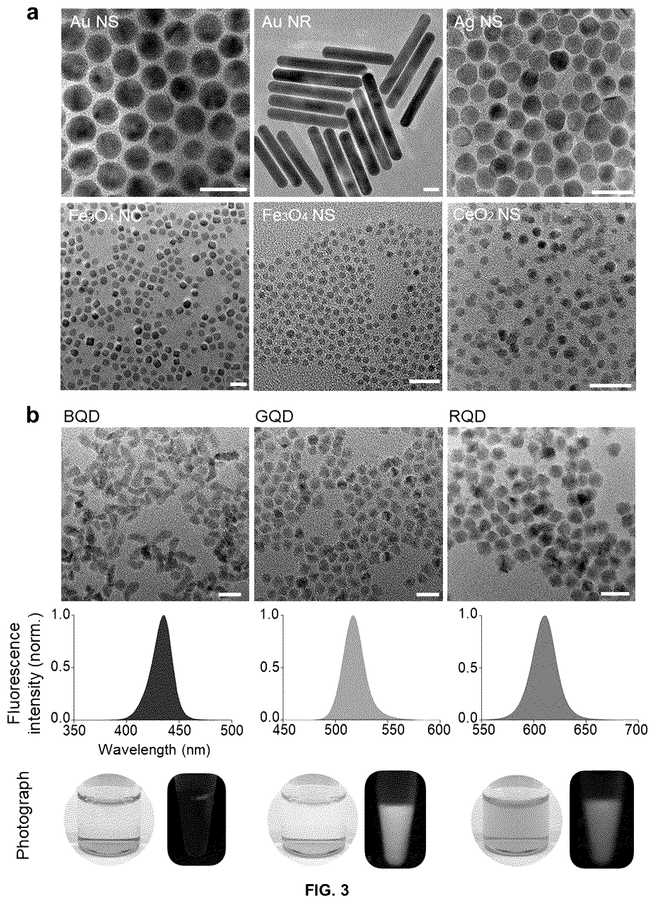

Surfactants are amphiphilic and demonstrate distinct interactions with MOFs and nanoparticles, respectively (Dederichs, T., Möller, M. & Weichold, O., Langmuir 2009, 25, 10501-10506; and Liu, X.-Y. et al., Nano Lett. 2020, 20, 1774-1780). We thus leveraged surfactants to mediate and guide nanoparticle integration into MOF hosts, to achieve precise control of nanoparticle organization and spatial distribution in STARs (FIG. 1a). Specifically, strong interactions between surfactants (primarily the polar heads) and MOF constituents drive heterogeneous MOF growth around the readily-incorporated surfactant molecules; weak interactions favor homogeneous MOF growth and tangential surfactant integration. On the other hand, strong interactions between surfactants (primarily the hydrophobic tails) and nanoparticles stabilize mono-dispersed nanoparticles while weak interactions induce nanoparticle aggregation. By rational selection of surfactants to tune and mediate these interactions, we designed and guided nanoparticle integration in STARs. FIG. 1b shows examples of the developed STARs. Through distinct surfactant modifications, gold (Au) nanoparticles were organized mono-dispersed and peripheral (FIG. 1b, left) or aggregated and central (FIG. 1b, right) with respect to the MOF structure. Importantly, the strategy can be applied to hosts with different crystallinity (e.g., 1D-oriented, 3D epitaxial and amorphous) (FIG. 2) and various nanoparticles (e.g., material composition, shape and size) (FIG. 3) to develop diverse STARs. The approach not only achieves fast, aqueous synthesis (<2 min at room temperature, one-pot reaction with water as the only solvent), but also enables in situ, templated growth on different solid substrates. Importantly, unlike conventional synthesis approaches which require harsh chemicals and lengthy processing, the STAR preparation is fast and safe (<2 min aqueous synthesis at room temperature). The technology can therefore be adopted to integrate sensitive, biological molecules (e.g., proteins and small molecule drugs) and achieve in situ growth on different types of solid substrates, to develop various multifunctional assemblies for diverse applications.

### Example 6. Surfactant Effects on STAR Assembly

In developing the STAR synthesis, we first evaluated the surfactant effects on MOFs and nanoparticles, respectively.

### Molecular Simulation

Molecular dynamics simulations between surfactants and MOF constituents were performed, in a surfactant-constituent pairwise manner, through a commercial software (Materials Studio 2018). We simulated interactions between the surfactant heads and the constituent molecules. For ionic surfactants, interactions were studied with and without counter ions. All molecular structures were modeled by sketch tools and geometrically optimized. The intermolecular interactions were simulated through the Quench task of Forcite module using the COMPASS force field with a microcanonical (NVE) ensemble. For each surfactant-constituent pair examined, three different molecular dynamics simulations were carried out: surfactant-constituent complex, surfactant only, and constituent only. The interaction energy (E_{s/c}) is calculated as E_{s/c}=E_{s+c}−(Eₛ+E_{c}), where E_{s+c}, Eₛ and E_{c} are the potential energies of surfactant-constituent complex, surfactant only, and constituent only, respectively. All simulations were set with the following parameters: temperature at 25° C., duration of 50 ps with a time step of 1 fs. Simulation data collected in the last 40 ps were used for structural and statistical analysis.

### Control of Nanoparticle Distribution in STAR

We utilized different surfactant modifications to tune and direct various nanoparticle assembly and distribution in different MOF hosts. To achieve the desired nanoparticle distribution, we selected respective surfactants, according to their interaction profiles as predicted by the molecular dynamics simulations. For the integration of a single type of nanoparticles, we regulated the loading ratio of different surfactant-modified nanoparticles to control their spatial distributions. For example, to prepare Au-ZIF-8, Au-Tween 20 and Au-CTAB (2 μl, 5 mg/ml) were independently prepared, and mixed with Zn²⁺ solution (50 μl, 0.05 M). The solutions were added in various proportions to HMIM (50 μl, 2.5 M). After vigorous mixing, the reaction was allowed to react in static for 10 min. The prepared composites were collected through centrifugation and characterized for spatial distribution. For the integration of multiple types of nanoparticles, to achieve the desired spatial distribution, we matched the nanoparticles with different surfactant modifications. For example, to prepare (Au, Fe₃O₄)-ZIF-8 composite with monodispersed Au locating peripherally (outside) and aggregated Fe₃O₄ encapsulated centrally (inside) of the MOF host, Au-Tween 20 and Fe₃O₄-CTAB (2 μl, 5 mg/ml) were independently prepared, mixed with Zn²⁺ solution (50 μl, 0.05 M), and added in various proportions to HMIM solution (50 μl, 2.5 M). After vigorous mixing, the reaction was allowed to react in static for 10 min. The prepared composites were collected through centrifugation and characterized for spatial distribution.

### Results and Discussion

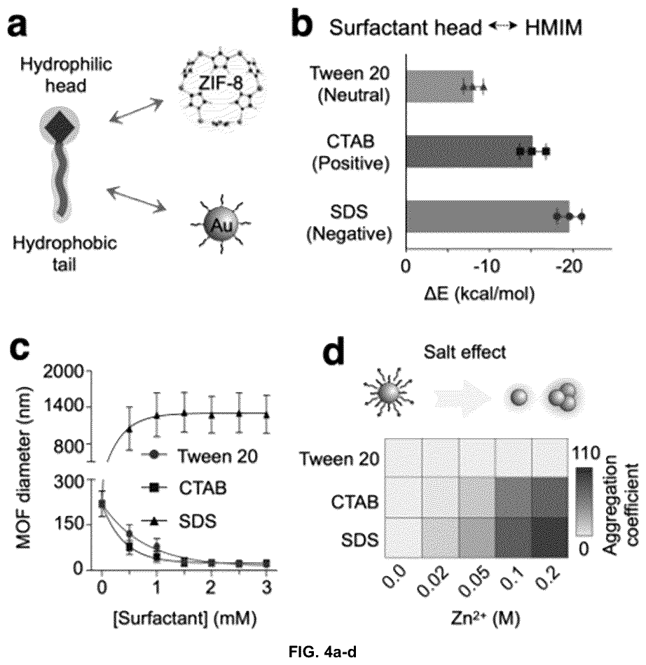

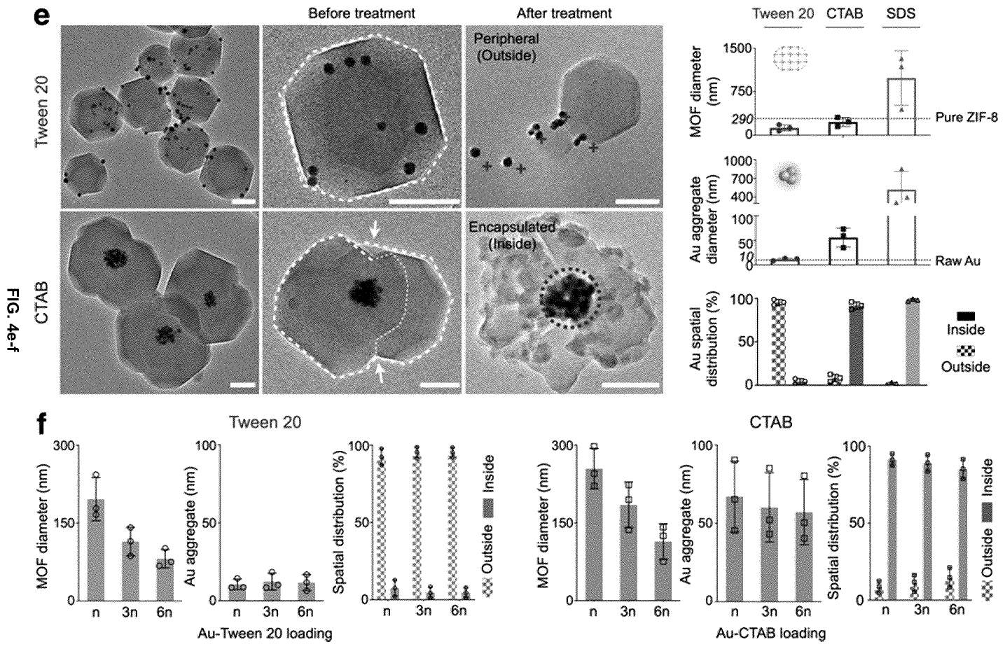

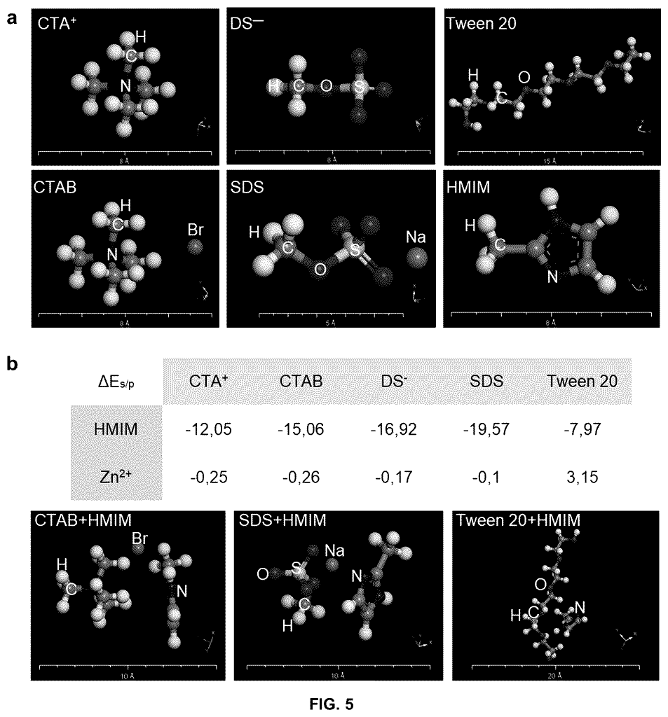

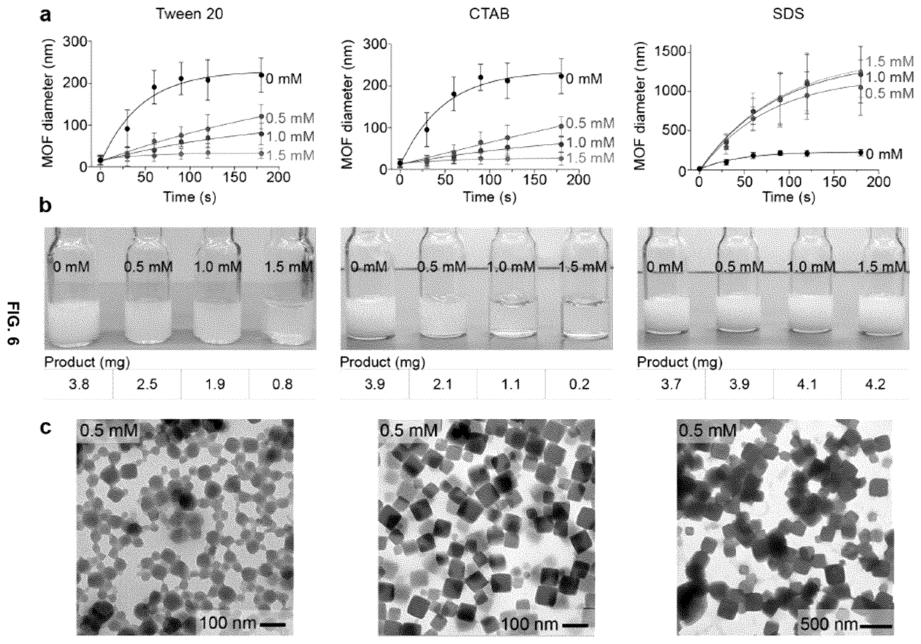

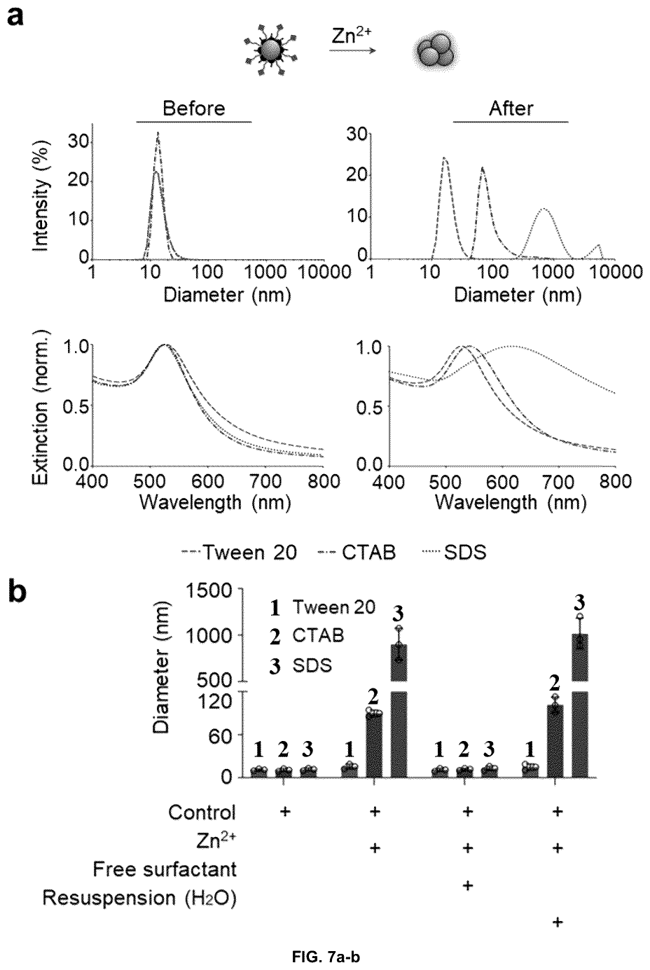

Using ZIF-8 and Au nanospheres as a model system (FIG. 4a), we selected three surfactant species with representative head properties and comparable tail properties, namely Tween 20 (neutral), CTAB (cationic) and SDS (anionic). Through molecular dynamics simulations, we profiled the interaction potentials (ΔE) of these surfactant heads with MOF constituents (2-methylimidazole/HMIM and Zn²⁺) (FIG. 5). The negatively charged SDS demonstrated the strongest interactions with the MOF constituents while the neutral Tween 20 showed the weakest interactions (SDS>CTAB>Tween 20) (FIG. 4b). Experimentally, the addition of SDS substantially increased the size of the prepared MOF, while CTAB and Tween 20 decreased the MOF size, in a dose-dependent and time-progressive manner (FIGS. 4c and 6). These empirical results are in agreement with the simulation data, suggesting that SDS as a strong interactor can be readily incorporated into and propagate MOF growth, while weak interactors such as Tween 20 reduce MOF growth. We next evaluated the efficacy of these surfactants in stabilizing nanoparticles. Surfactant-coated Au nanospheres were incubated with an increasing concentration of Zn²⁺ (a ZIF-8 constituent) (FIG. 4d). Among the tested surfactants, Tween 20 offered the strongest stabilization while surfactants with a charged head group (CTAB and SDS) induced extensive nanoparticle aggregation, indicating different surfactant susceptibilities to the salt effects induced by MOF constituents (Qazi, M. J. et al., Langmuir 2017, 33, 4260-4268, FIG. 7a-c). For surfactants with a similar head property but different tail lengths (e.g., CTAB vs. DTAB), the longer tail-surfactant provided better nanoparticle stabilization (FIG. 7d), likely due to its stronger hydrophobic interaction with nanoparticles (Atkin, R. et al., Adv. Colloid Interface Sci. 2003, 103, 219-304).

### Example 7. Surfactant Effects in Preparing Different STARs

We next investigated the surfactant effects in preparing different STARs. We prepared Au nanospheres with different surfactant coating before mixing them with MOF constituents (HMIM and Zn²⁺) by following the protocol in Example 4.

### Analysis of Nanoparticle Spatial Distribution

To quantify nanoparticle concentration in the as-synthesized STAR assembly (Nₜₒₜₐₗ), we measured its elemental content by inductively coupled plasma-optical emission spectrometry (ICP-OES) (Perkin Elmer Avio 500). The nanoparticle spatial distribution in MOF was evaluated through in situ washes, as described in Example 5. As the wash buffer infiltrates the MOF structure, it first dislodges the peripherally-associated nanoparticles (outside) from the MOF host; the centrally-encapsulated nanoparticles (inside) remain within the MOF host. The wash conditions were optimized through TEM characterization to achieve clear differentiation of the outside population: acidic HCl buffers (pH=4.0) were used for HMIM-based and BTC-based samples, and alkaline NaOH buffers (pH=9.0) were used for BDC-based samples. All wash incubations were kept to 5 min. To quantify nanoparticles dislodged into the supernatant (N_{outside}), after in situ washes, we recovered the supernatant and quantified its elemental content through ICP-OES.

The nanoparticle spatial distribution in STAR assembly is determined as below:

ρ_{outside}=N_{outside}/Nₜₒₜₐₗ

ρ_{inside}=1−ρ_{outside}

Where ρ_{outside} and ρ_{inside} are fractions of nanoparticles distributed outside and inside of the STAR assembly, respectively. Nₜₒₜₐₗ is the total number of nanoparticles in the STAR assembly.

N_{outside} is the number of peripherally-associated nanoparticles, dislodged into the supernatant after in situ washes.

### Results and Discussion

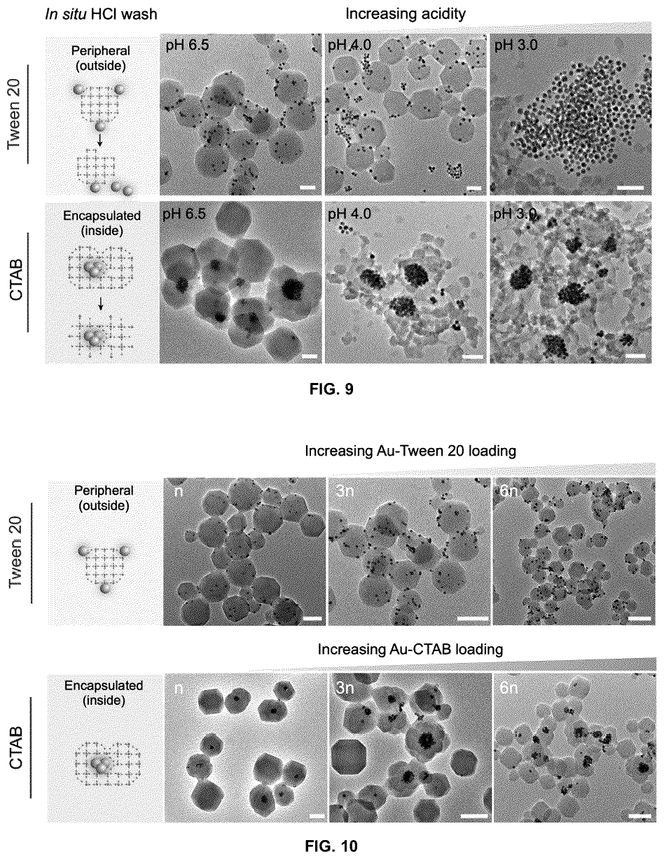

In all cases, STARs formed rapidly upon reagent mixing (FIG. 8a). TEM confirmed architectural differences (e.g., nanoparticle size and distribution) in these STARs (FIG. 4e), consistent with predictions made with respect to various surfactant effects on MOF growth and nanoparticle stability (FIG. 8b). Specifically, with Tween 20 coating, mono-dispersed nanoparticles were peripherally associated with intact MOF hosts; the formed structures showed a small diameter and regular features (outlined by the dashed line). With CTAB coating, nanoparticle aggregates were encapsulated within the MOF structures; the formed STARs showed an increased diameter and structural distortions (indicated by the arrows). SDS, on the other hand, resulted in big nanoparticle aggregates encapsulated within large, irregular MOFs (FIG. 8c). All nanoparticle spatial distributions within STARs were confirmed through in situ acid washes (FIG. 9). While Tween 20-coated nanoparticles could be readily dislodged from the formed STARs, CTAB-coated nanoparticles remained encapsulated, even under harsh acid wash conditions. Interestingly, by tuning the nanoparticle loading concentration (FIG. 10), different-sized STARs with consistent nanoparticle organization and distribution could be prepared (FIG. 4f). We thus focused all subsequent STAR development using Tween 20 and CTAB as the guiding surfactants.

### Example 8. Tunable STAR Development

To evaluate the versatility of the surfactant-guided assembly, we expanded the strategy in Example 4 to prepare different STARs using diverse nanoparticles of different sizes, shapes and materials with various MOF hosts, including another 3D epitaxial MOF (ZIF-67), 1D-oriented MOFs (Cu-BDC, Cu-BTC, Ce-BDC and Ce-BTC) as well as amorphous products (Fe-BDC and Fe-BTC). Nanoparticle spatial distribution with respect to the MOF host was performed by following the protocol in Example 7.

### Results and Discussion

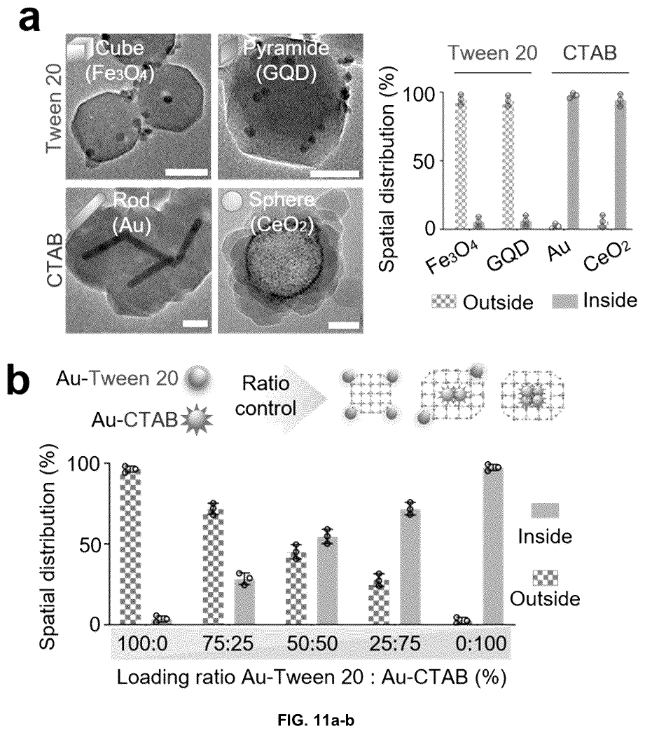

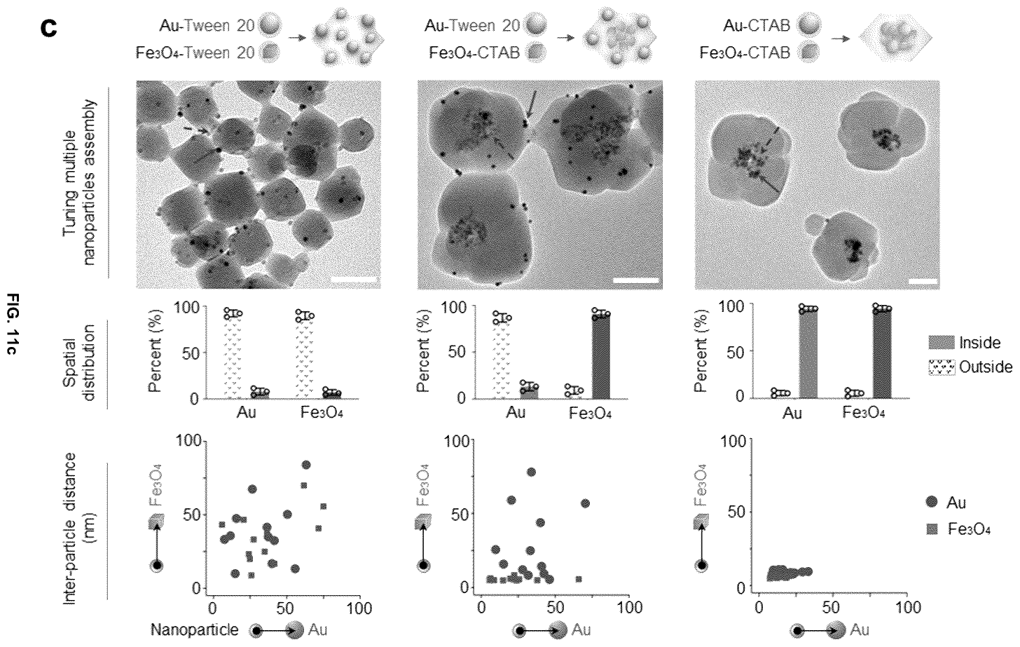

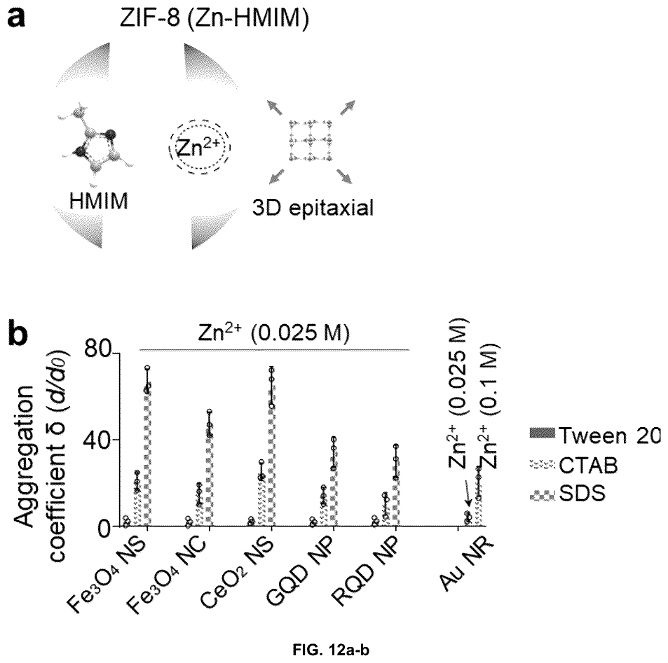

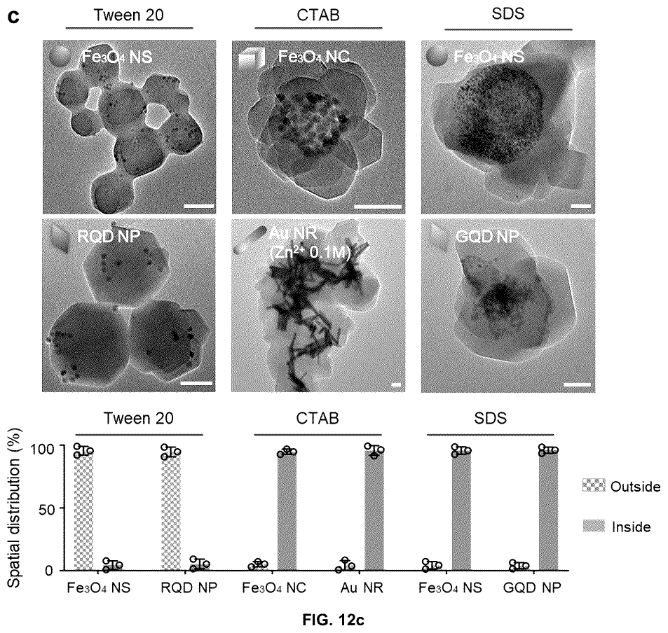

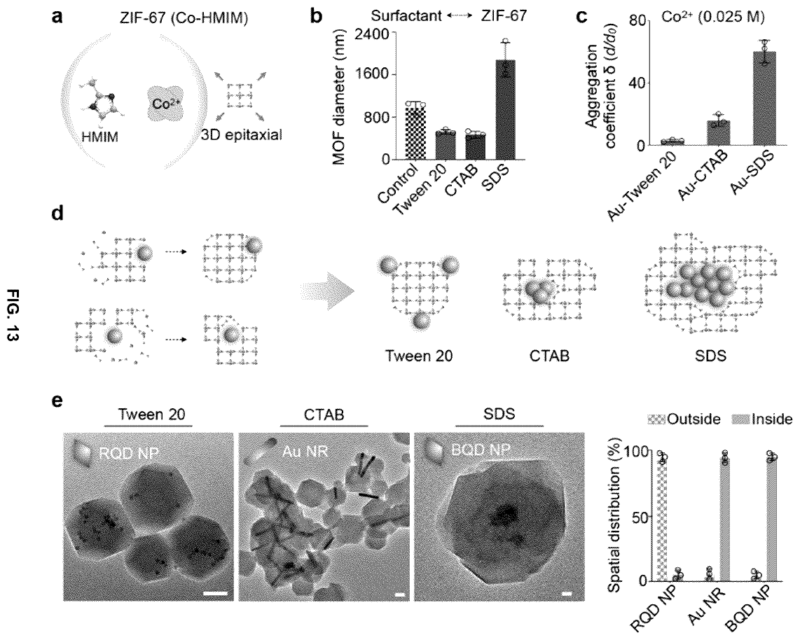

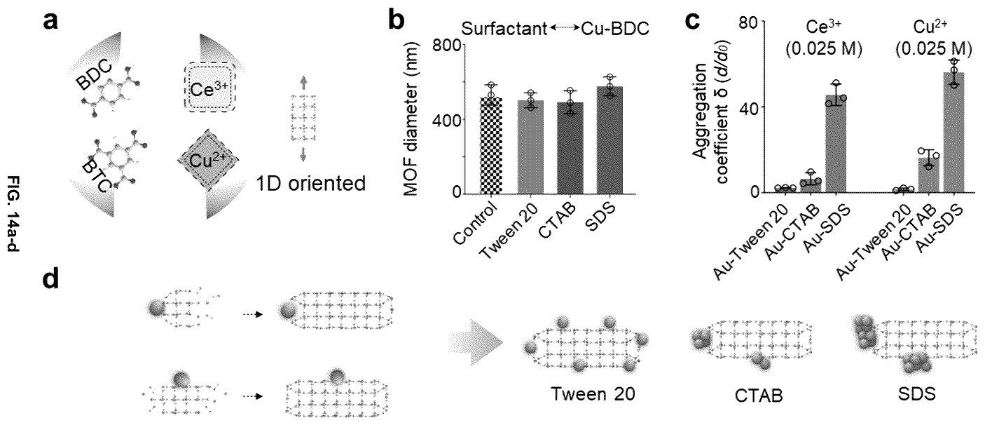

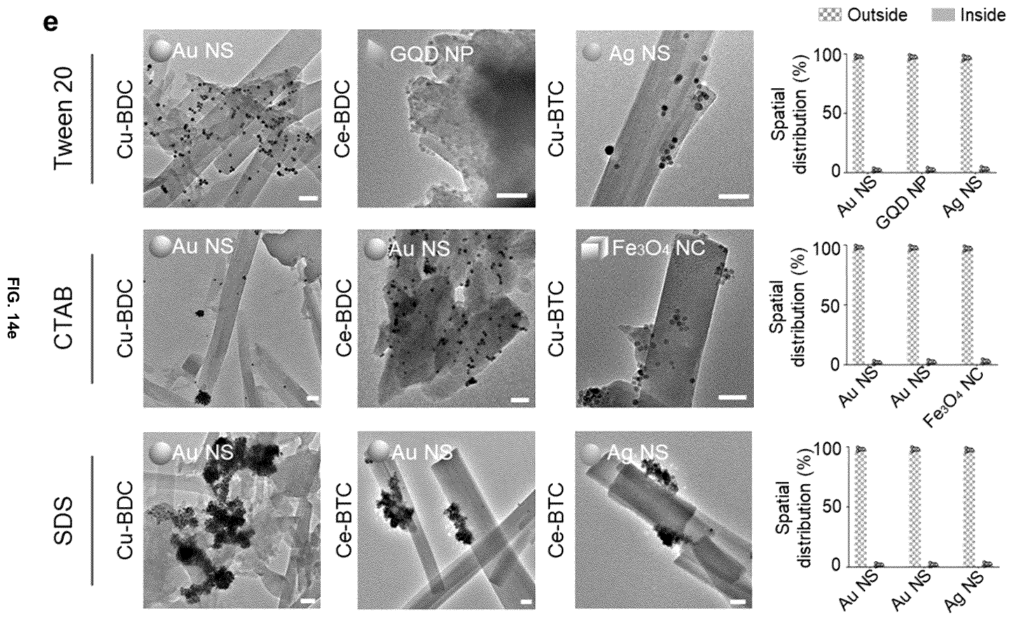

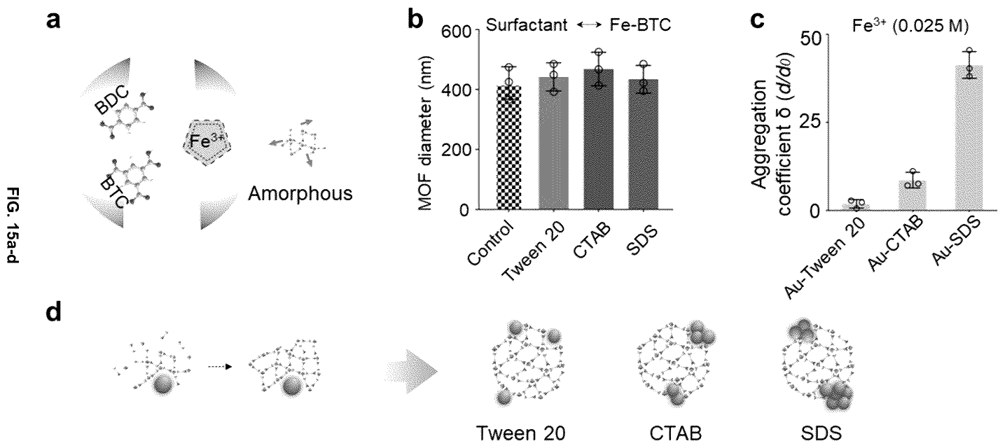

TEM characterization verified that across all tested nanomaterials, the organization and distribution of nanoparticles within the STARs were consistent with the predicted architectures (FIGS. 11a and 12). The strategy in Example 4 was further expanded to integrate nanoparticles in other MOF systems, including another 3D epitaxial MOF (ZIF-67) (FIG. 13), 1D-oriented MOFs (Cu-BDC, Cu-BTC, Ce-BDC and Ce-BTC (FIG. 14) as well as amorphous products (Fe-BDC and Fe-BTC) (FIG. 15). Systematic characterization of the resultant STARs confirmed that all nanoparticle integration (i.e., spatial organization and distribution) abide by the predications as determined by surfactant interactions.

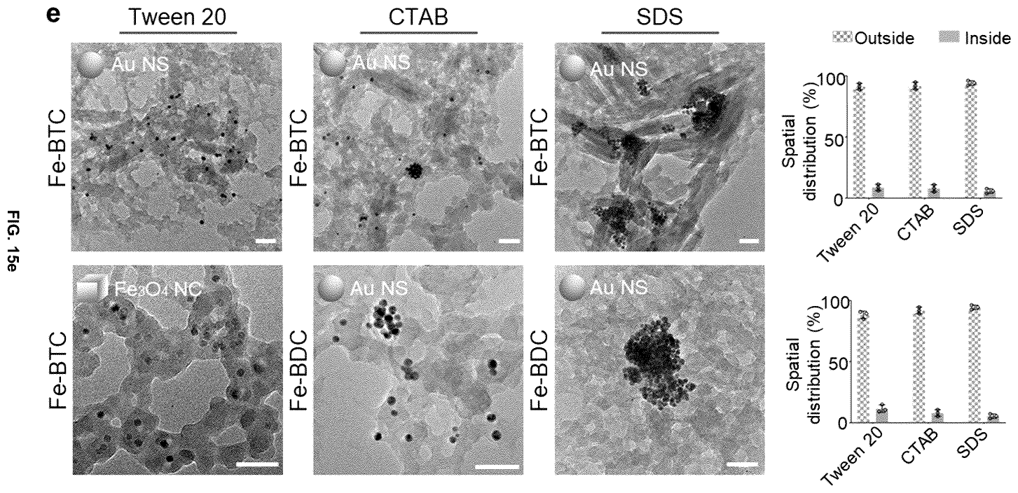

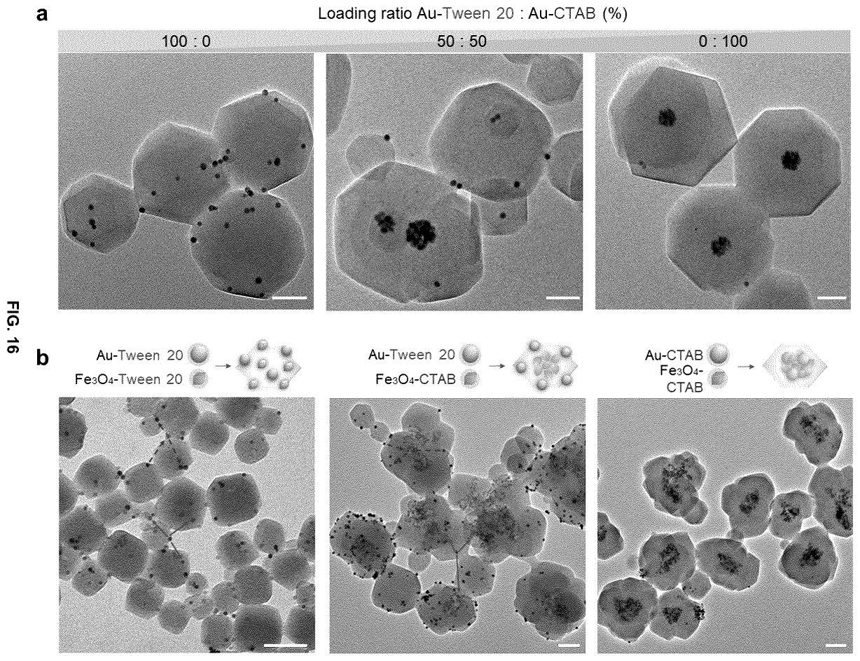

Inspired by its universality, we exploited the approach to design and develop complex architectures. Using Au nanospheres coated with Tween 20 and CTAB, respectively, we varied the loading ratio of these nanoparticle populations. The approach achieved precise spatial tuning of nanoparticle distribution within individual MOFs; the STAR morphology correlated well with the initial nanoparticle loading ratio and matched closely to the designed architecture (FIG. 11b). Specifically, with increased loading of Tween 20-coated nanoparticles, more particles resided on the MOF periphery; with increased loading of CTAB-coated nanoparticles, more nanoparticle aggregates were observed encapsulated within the MOFs (FIG. 16a).

We next employed this approach of surfactant matching to precisely integrate and distribute different types of nanoparticles in STARs. Using Au and Fe₃O₄ nanoparticles of different sizes and shapes, we coated them with Tween 20 and CTAB to develop multi-particle architectures (FIG. 11c, top and 16b). Nanoparticle spatial distribution (with respect to the MOF host, FIG. 11c, middle) was quantified through in situ acid washes. We further measured inter-nanoparticle distance, through TEM characterization, to reflect nanoparticle organization (with respect to other nanoparticles, FIG. 11c, bottom). The analyses confirmed the efficacy of surfactant matching in tuning particle distribution and organization: (i) with universal Tween 20 coating, all nanoparticles (Au: sphere; Fe₃O₄: cube) were peripherally associated with the MOF host and remained dispersed; (ii) when applied as a mixture, nanoparticles distributed and organized according to their surfactant coating. Tween 20-coated Au nanoparticles were peripherally located and remained dispersed, while CTAB-coated Fe₃O₄ nanoparticles were encapsulated and aggregated within MOFs; and (iii) with universal CTAB coating, all nanoparticles were aggregated and located centrally within MOFs.

### Example 9. Rapid Assembly for Biotechnology Applications

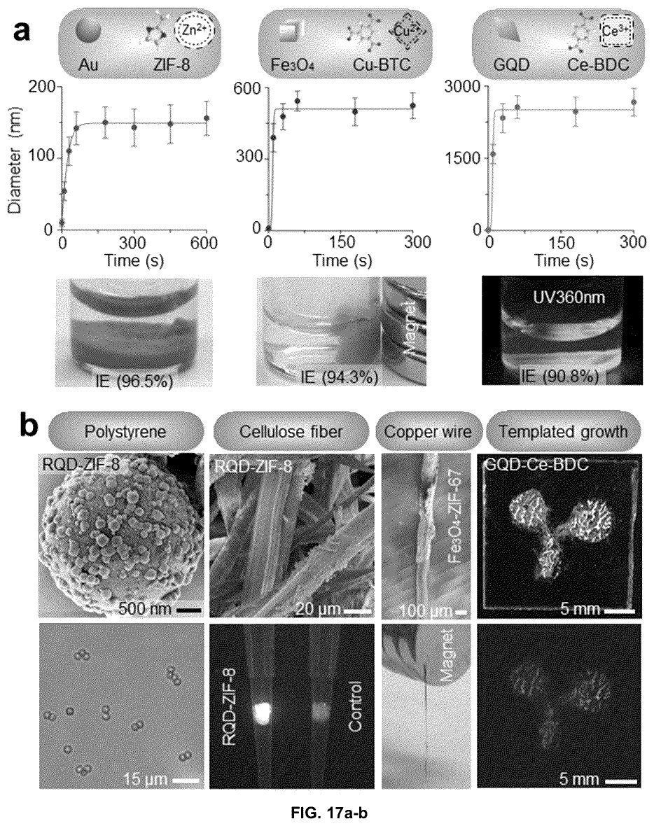

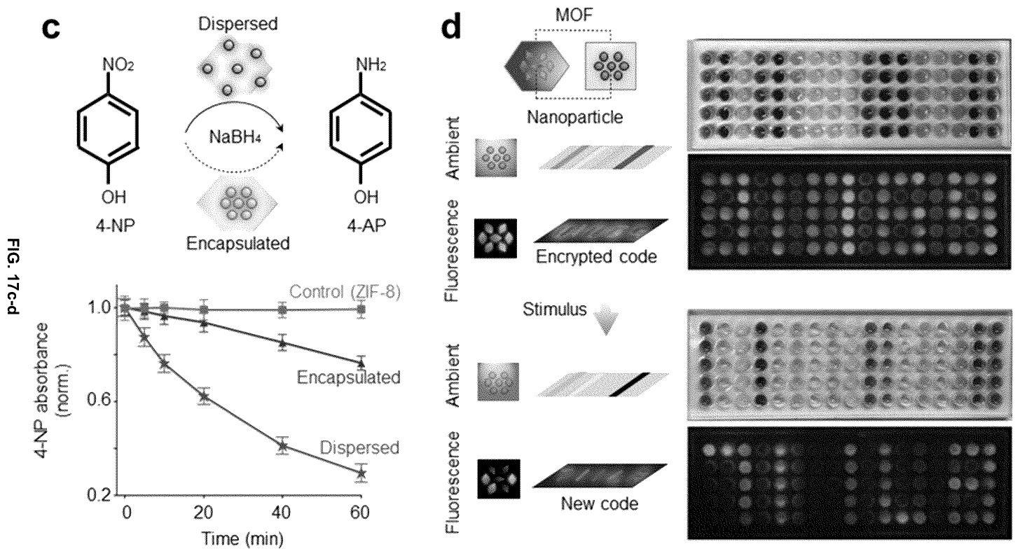

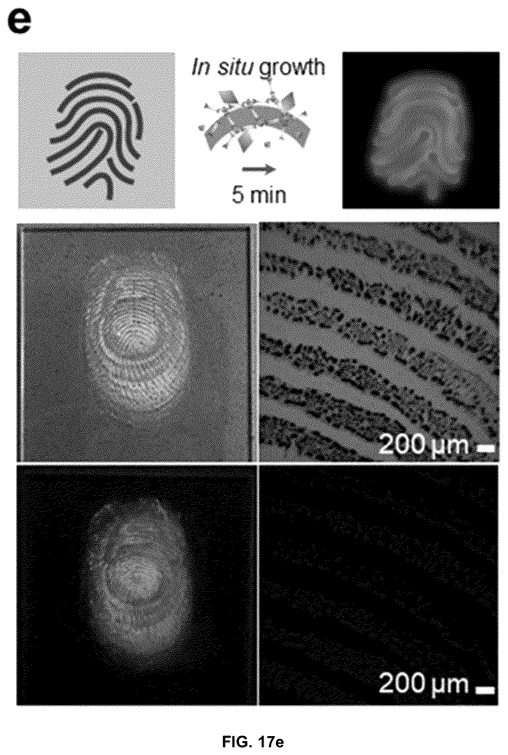

To apply STARs for various biotechnology applications, we first evaluated the robustness of their preparation in solution and on solid substrates. Using different nanoparticles and MOF hosts, we prepared STARs as a suspension through aqueous synthesis (FIG. 17a) by following the protocol in Example 4.

### Cellular Toxicity of STAR Composites

To evaluate cellular toxicity, we employed the MTS cell proliferation assay (Thermo Scientific). Per manufacturer's protocol, epithelial cells (A431) were seeded and incubated with different concentrations of STAR composites for 24 h. After the addition of MTS reagent, absorbance (490 nm) was measured to evaluate cell viability (Tecan).

### STAR Incorporation of Biomolecules

To integrate DOX and BSA into the RQD-ZIF-8 STAR assembly, Tween 20-stabilized RQD nanoparticles (5 mg/ml, 2.5 μl) were mixed with Zn(NO₃)₂ (0.05 M, 50 μl), followed by the sequential addition of BSA (10 mg/ml, 4 μl), DOX (10 mg/ml, 2 μl), and HMIM (2.5 M, 50 μl). After vigorous mixing, the mixture was allowed to react in static for 10 min. All solutions were prepared with distilled water.

### In Situ Growth on Substrates

STAR assemblies were grown in situ on various substrates at room temperature. Briefly, surfactant-modified nanoparticles, metal node solution and organic linker solution were mixed and loaded immediately onto substrates (e.g., polystyrene, metal, cellulose, glass). After 10 min of incubation, the substrates were washed in water to remove unbound composites and dried for further characterization. The synthesis precursors for different substrates are as follows: polystyrene beads: Zn²⁺ node (50 mM, 0.5 ml), HMIM (2.5 M, 0.5 ml), RQD-Tween 20 (5 mg/ml, 25 μl); copper wire: Co²⁺ node (50 mM, 0.5 ml), HMIM (2.5 M, 0.5 ml), Fe₃O₄ nanocube-SDS (5 mg/ml, 25 μl); cellulose fibre: Zn²⁺ node (25 mM, 0.5 ml), HMIM (2.5 M, 0.5 ml), RQD-Tween 20 (5 mg/ml, 25 μl); glass slide: Ce³⁺ node (25 mM, 0.5 ml), BDC (25 mM, 0.5 ml), GQD-Tween 20 (5 mg/ml, 25 μl).

### Results and Discussion

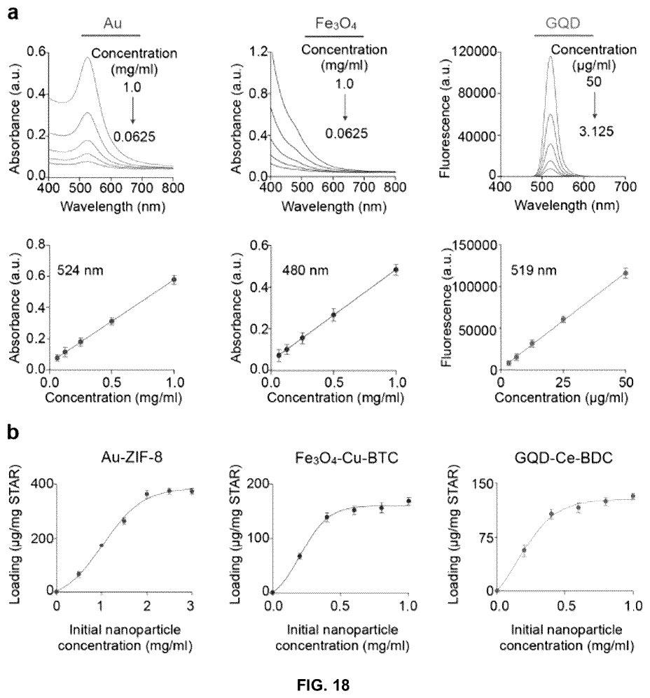

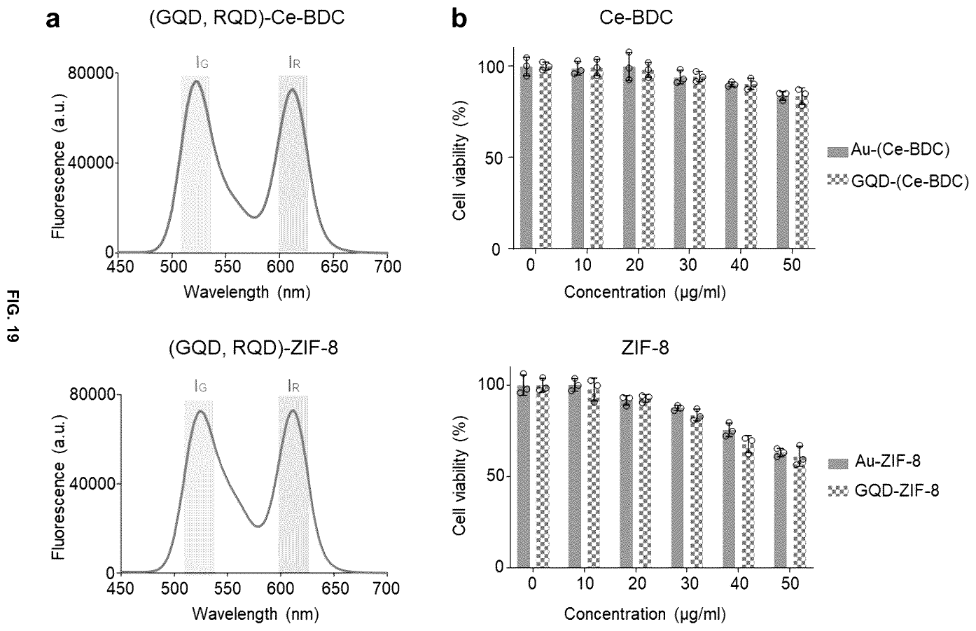

The reaction could be completed in <2 min at room temperature, with water as the only solvent. Importantly, the reaction achieved controlled nanoparticle loading and yielded a high nanoparticle integration efficiency (>90%) to form composites with different properties (FIG. 18). For composites developed with identical nanoparticles in different MOF hosts, the choice of MOF demonstrates a small influence on the nanoparticle-endowed properties (e.g., fluorescence) but a strong effect on the cellular toxicity of the composites (FIG. 19). In addition to inorganic nanoparticles, this aqueous synthesis also enhanced the integration of biological molecules (e.g., proteins and small molecule drugs) (FIG. 20).

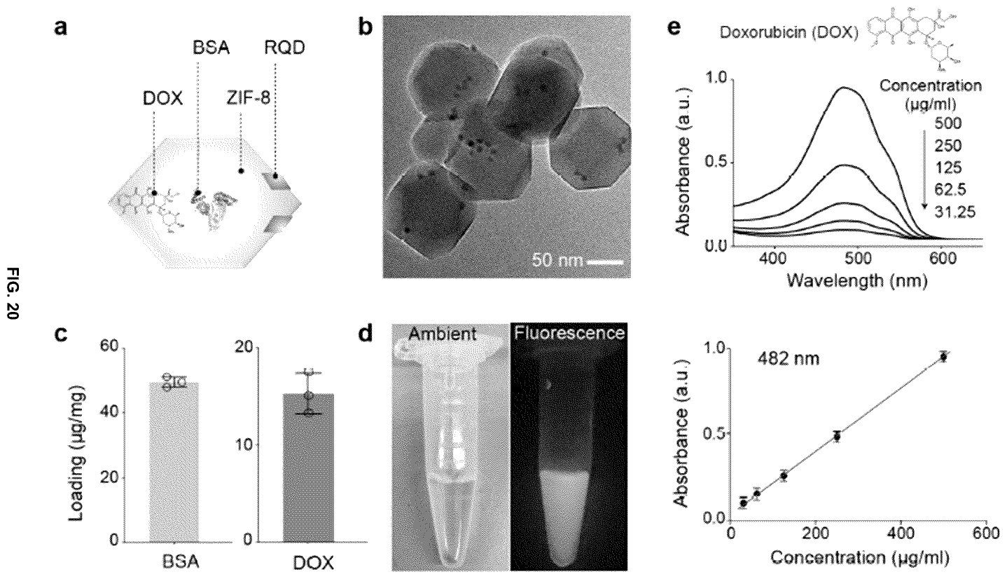

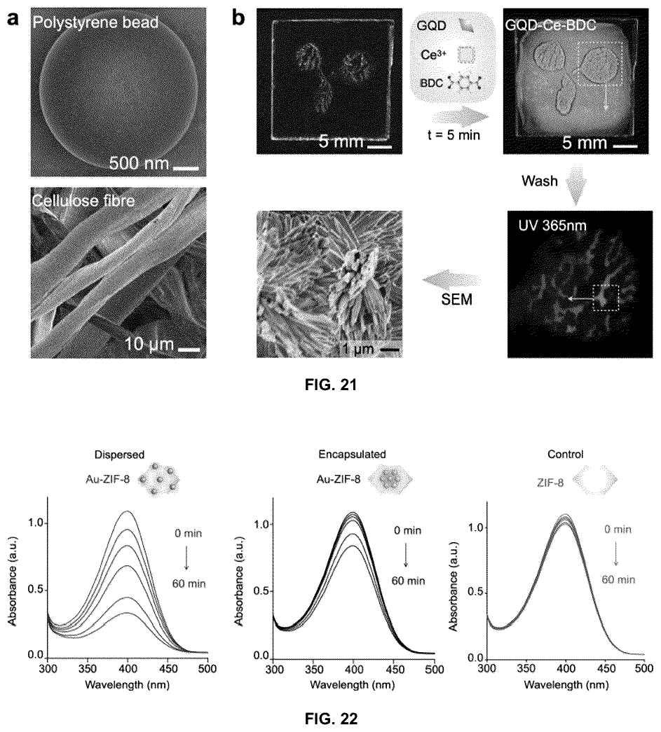

We further assessed the assembly of STARs on various solid substrates (e.g., polystyrene microspheres, cellulose mesh, copper wire) (FIGS. 17b and 21). Through multimodal characterization, we not only observed rapid, in situ STAR formation on different solid supports, but also confirmed its templated growth on treated surfaces, with minimal development in the control regions. Therefore, this approach not only tunes the overall morphology but also achieves intra-assembly spatial control of nanoparticle organization and distribution to develop hybrid architectures.

### Example 10. Application of STARs for Various Biotechnology Applications

We next applied STARs, prepared in solution or on substrates, for various biotechnology applications. Using differentially-coated Au nanoparticles and ZIF-8 as a host, we prepared STARs with varied spatial distribution of nanoparticles, dispersed vs. encapsulated. We applied these two types of STARs as nanocatalysts for the reduction of 4-NP to 4-AP (FIG. 17c, Li, M. & Chen, G., Nanoscale 2013, 5, 11919-11927).

### STAR for Catalysis

Two Au-ZIF-8 composites with distinct Au nanoparticle distributions (i.e., peripherally dispersed Au-Tween 20 and encapsulated Au-CTAB) were adopted as catalysts for the conversion of 4-NP to 4-AP in the presence of reductive NaBH₄. The Au-ZIF-8 composites were synthesized using Au nanoparticles (2.5 μl, 5 mg/ml), Zn(NO₃)₂ (50 μl, 0.05 M), and HMIM (50 μl, 2.5 M) by following the protocol in Example 4. For the analysis of catalytic efficiency, 4-NP (0.25 ml, 1 mM, pH=10) and freshly prepared aqueous NaBH₄ (0.25 ml, 50 mM) were mixed in 3.5 ml of water. Subsequently, Au-ZIF-8 composites (0.1 ml, 5 mg/ml) were added to the reaction. UV-Vis absorption spectra were recorded in real time to monitor the concentration of 4-NP (Tecan).

### STAR for Encryption

A PMMA array with 5×17 wells was prepared via a tabletop CO₂ laser engraver (Universal Laser Systems). We employed microscopic (STAR assemblies) and macroscopic patterning (well positioning) to encrypt information (Table 1). For microscopic encryption, various STAR assemblies were synthesized; these assemblies contain combinations of nanoparticles, differentially distributed in various MOF hosts. For macroscopic patterning, different STAR assemblies were mixed with polyacrylamide gel precursor (4% PAGE gel, Bio-Rad) and allowed to polymerize in defined wells. To achieve information transformation, the device was treated with 2% acetic acid as the stimulus for 10 min, before being washed in water, to reveal the encrypted code.

TABLE 1
STAR composition and arrangement in the encryption device.

| | 1 | 2 | 3 | 4 | 5 |
|---|---|---|---|---|---|
| A | (RQD-Tween20, | 1. (RQD-Tween20, | 1. (RQD-Tween20, | 1. (RQD-Tween20, | 1. (RQD-Tween20, |
| | Au-Tween20)- | Au-Tween20)- | Au-Tween20)- | Au-Tween20)- | Au-Tween20)- |
| | Ce-BDC | Ce-BDC(95%) | Ce-BDC(95%) | Ce-BDC(95%) | Ce-BDC(95%) |
| | | 2. Cu-BTC(5%) | 2. Cu-BTC(5%) | 2. Cu-BTC(5%) | 2. Cu-BTC(5%) |
| B | GQD-Tween20- | ZIF-67 | GQD-Tween20- | ZIF-67 | GQD-Tween20- |
| | ZIF-67 | | ZIF-67 | | ZIF-67 |
| C | RQD-Tween20- | RQD-Tween20- | RQD-Tween20- | RQD-Tween20- | RQD-Tween20- |
| | Ce-BDC | Ce-BDC | Ce-BDC | Ce-BDC | Ce-BDC |
| D | Au-SDS-ZIF-8 | Au-SDS-ZIF-8 | Au-SDS-ZIF-8 | Au-SDS-ZIF-8 | Au-SDS-ZIF-8 |
| E | GQD-Tween20- | GQD-Tween20- | GQD-Tween20- | GQD-Tween20- | GQD-Tween20- |
| | ZIF-67 | ZIF-67 | ZIF-67 | ZIF-67 | ZIF-67 |
| F | Fe-BDC | Fe-BDC | Fe-BDC | Fe-BDC | Fe-BDC |
| G | 1. Cu-BTC(70%) | 1. Cu-BTC(70%) | 1. Cu-BTC(70%) | 1. Cu-BTC(70%) | 1. Cu-BTC(70%) |
| | 2. GQD-Tween20- | 2. GQD-Tween20- | 2. GQD-Tween20- | 2. GQD-Tween20- | 2. GQD-Tween20- |
| | Ce-BDC(30%) | Ce-BDC(30%) | Ce-BDC(30%) | Ce-BDC(30%) | Ce-BDC(30%) |
| H | 1. Cu-BTC(60%) | Cu-BTC | Cu-BTC | Cu-BTC | 1. Cu-BTC(60%) |
| | 2. GQD-Tween20- | | | | 2. GQD-Tween20- |
| | Ce-BDC(40%) | | | | Ce-BDC(40%) |
| I | RQD-Tween20- | RQD-Tween20- | RQD-Tween20- | RQD-Tween20- | RQD-Tween20- |
| | Ce-BDC | Ce-BDC | Ce-BDC | Ce-BDC | Ce-BDC |
| J | Au-Tween20- | Au-Tween20- | Au-Tween20- | Au-Tween20- | Au-Tween20- |
| | Ce-BDC | Ce-BDC | Ce-BDC | Ce-BDC | Ce-BDC |
| K | (GQD-Tween20, | (GQD-Tween20, | (GQD-Tween20, | (GQD-Tween20, | (GQD-Tween20, |
| | Au-Tween20)- | Au-Tween20)- | Au-Tween20)- | Au-Tween20)- | Au-Tween20)- |
| | ZIF-67 | ZIF-67 | ZIF-67 | ZIF-67 | ZIF-67 |
| L | 1. (GQD-Tween20, | ZIF-67 | 1. (GQD-Tween20, | ZIF-67 | (GQD-Tween20, |
| | Au-Tween20)- | | Au-Tween20)- | | Au-Tween20)- |
| | ZIF-67 | | ZIF-67 | | ZIF-67 |
| | 2. Cu-BTC(5%) | | 2. Cu-BTC(5%) | | |
| M | 1. Fe-BDC(50%) | Fe-BDC | 1. Fe-BDC(50%) | 1. Fe-BDC(50%) | 1. Fe-BDC(50%) |
| | 2. RQD-Tween20- | | 2. RQD-Tween20- | 2. RQD-Tween20- | 2. RQD-Tween20- |
| | Ce-BDC(45%) | | Ce-BDC(45%) | Ce-BDC(45%) | Ce-BDC(50%) |
| | 3. Cu-BTC(5%) | | 3. Cu-BTC(5%) | 3. Cu-BTC(5%) | |
| N | Cu-BTC | Cu-BTC | Cu-BTC | Cu-BTC | Cu-BTC |
| O | 1. Fe-BDC(50%) | 1. Fe-BDC(50%) | 1. Fe-BDC(50%) | Fe-BDC | 1. Fe-BDC(50%) |
| | 2. RQD-Tween20- | 2. RQD-Tween20- | 2. RQD-Tween20- | | 2. RQD-Tween20- |
| | Ce-BDC(50%) | Ce-BDC(45%) | Ce-BDC(50%) | | Ce-BDC(50%) |
| | | 3. Cu-BTC(5%) | | | |
| P | (GQD-Tween20, | Au-SDS-ZIF-67 | (GQD-Tween20, | Au-SDS-ZIF-67 | (GQD-Tween20, |
| | Au-SDS)-ZIF-67 | | Au-SDS)-ZIF-67 | | Au-SDS)-ZIF-67 |
| Q | (RQD-Tween20, | (RQD-Tween20, | (RQD-Tween20, | (RQD-Tween20, | (RQD-Tween20, |
| | Au-Tween20)- | Au-Tween20)- | Au-Tween20)- | Au-Tween20)- | Au-Tween20)- |
| | Ce-BDC | Ce-BDC | Ce-BDC | Ce-BDC | Ce-BDC |

STAR loading in each well is 0.5 mg. Number in parenthesis denotes weight percentage.

### STAR for Fingerprint Detection

To reveal latent fingerprints on a solid surface, MOF constituents (Ce³⁺ and BDC) and RQD stabilized by Tween 20 were applied immediately onto the surface. Specifically, a reaction mixture comprising Ce³⁺ node (25 mM), BDC (25 mM), and RQD-Tween 20 (5 mg/ml), mixed in a volumetric ratio of 100:100:1, was applied to the surface. After a quick incubation (5 min at room temperature), the unbound materials were flushed with water. The fingerprint-induced STAR patterning could be visualized under UV excitation (365 nm).

### Results and Discussion

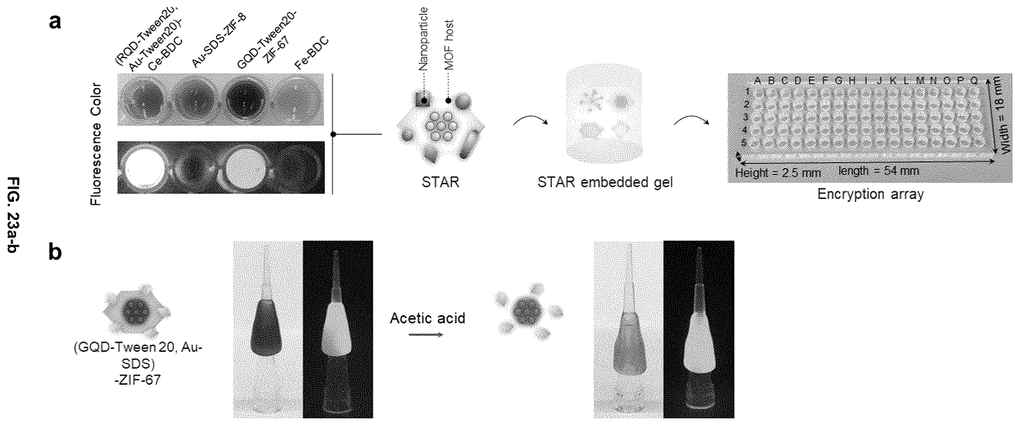

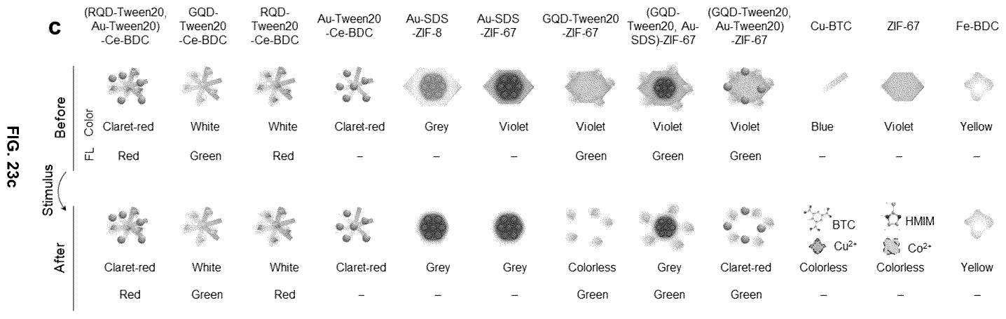

By absorbance measurement, we demonstrated that the dispersed STARs have a higher catalytic efficiency than the encapsulated form (FIG. 22). Second, using different combinations of STARs, we developed an encryption array (FIG. 17d). Due to their material composition (e.g., MOFs and nanoparticles), these STARs exhibit different optical properties (e.g., color under ambient lighting and fluorescence under UV excitation); due to their intra-assembly nanoparticle spatial distribution, they respond differently to external stimuli (e.g., acetic acid) to generate different optical signals (FIG. 23). By embedding different STARs in a polyacrylamide array (Table 1), we encoded information and employed the STARs' different responses (i.e., color and fluorescence changes, in response to stimulus) to reveal the encrypted code. Lastly, we applied the in situ development of STARs to reveal latent fingerprints (FIG. 17e). Motivated by its templated growth along biomolecule-treated surfaces (e.g., lipids, which are commonly found in human fingerprints, FIG. 21b), we leveraged the STAR assembly to achieve rapid and direct visualization of latent fingerprints.

### Example 11. Development of Complementary STARs for Various Biotechnology Applications

Drawing on the advantages above, we developed complementary STARs for various biotechnology applications. In particular, we developed a dual-probe STAR to achieve direct profiling of extracellular vesicle (EV) glycans in clinical specimens (FIG. 1c). Using different surfactants, we spatially organized two types of QD probes within the MOF assembly (FIG. 3b). Their relative spatial distribution within the MOF host endowed the probes with different responsiveness; probes located peripheral in the formed STAR readily react with external stimuli while probes found central serve as intrinsic references (i.e., peripheral working probes vs. central reference probes). We employed this positional selectivity to achieve sensitive and specific biomarker detection. In the presence of target EV glycan, the specific binding of lectin-oxidase mediates in situ generation of H₂O₂, which selectively quenches the fluorescence of the peripheral working probes, while leaving the central reference probes in the same STAR assemblies unaffected.

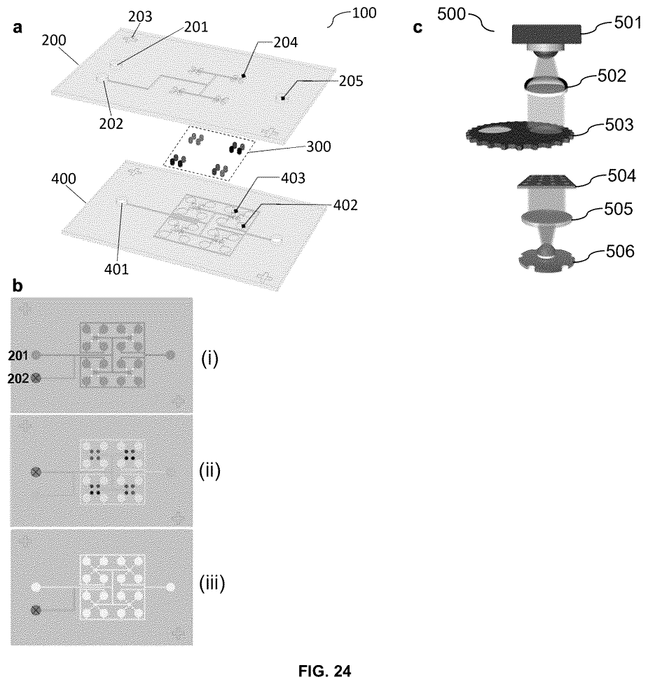

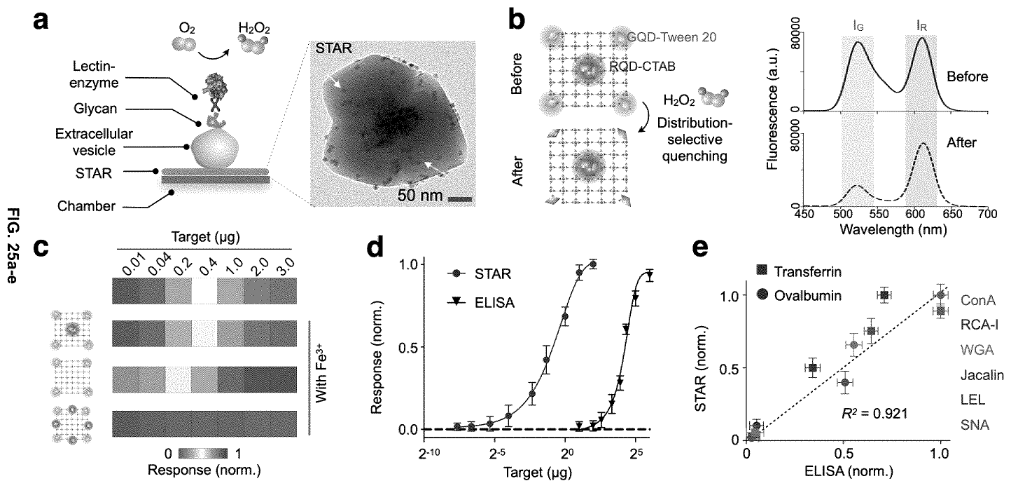

To streamline the assay workflow, we synthesized the STARs in a miniaturized microfluidic device and performed EV glycan measurements on-chip (FIG. 1d). The device consists of a lower channel 400, where STARs are grown in situ, and an upper channel 200, which is preloaded with lectins (FIG. 24a). Solution mixing between the two channels is only actuated during assay operation to enable specific EV targeting and signal generation (FIG. 24b). Importantly, each chip comprises 16 parallel and independent detection chambers, and can be loaded onto a custom-designed, smartphone-based optical platform for multiplexed fluorescence measurements (FIG. 24c).

### Microfluidic Platform Fabrication

A prototype dual-layer microfluidic device (FIG. 24a) was fabricated through standard soft lithography. Briefly, SU-8 negative photoresist (SU8-2025, Microchem) was used to prepare the cast molds. The photoresist was spin-coated onto a silicon wafer and patterned with photomasks using a cleanroom mask aligner (SUSS MicroTec). After UV light exposure, the cast molds were developed under agitation. The molds were chemically treated with trichlorosilane vapor inside a desiccator, and then a mixture of PDMS and crosslinker at a ratio of 10:1 was casted onto the molds and cured at 80° C. The obtained PDMS replicas and glass substrate were aligned and surface bonded together after the plasma treatment, producing the microchannel. To enable in situ STAR growth in the bottom detection chamber, 5 μl of synthesis solution comprising Zn²⁺ (25 mM), HMIM (1.25 M), GQD-Tween 20 (0.5 mg/ml), RQD-CTAB (0.5 mg/ml) was introduced into the device. After 10 min incubation, the device was flushed with water to remove unbound composites. To confine lectins to the top chambers, lectins were dissolved in PBS (15 μg/ml) with excipient (PEG 2000, 1 mg/ml), preloaded into the defined chambers of the upper channel layer and lyophilized before PDMS bonding.

### STAR Assay for Glycosylation Profiling

Operation steps are illustrated in FIG. 24b. In a typical procedure, 5 μl of sample was introduced into the detection chambers. After 5 min incubation, the chambers were blocked by BSA (2% w/v) for 5 min. PBS buffer was introduced to flow through the top chambers, dissolving the preloaded lectins. The lectin solution was further driven into the bottom detection chambers, allowing the biotinylated lectins to bind to target glycan moieties for 5 min. Next, streptavidin-conjugated glucose oxidase (GOD) solution (10 μg/ml) was introduced into the detection chambers and incubated for 5 min. Glucose solution (2 mg/ml) was then added, to enable GOD oxidation of glucose to generate H₂O₂; this liberated H₂O₂, produced only in the presence of specific bound lectins, selectively quenches peripherally distributed GQD (outside) in the MOF host, but does not affect the centrally encapsulated RQD (inside). After a 10 min reaction, fluorescence measurements were performed to profile glycosylation.

All fluorescence signals are calculated relative to the central reference population (RQD):

$$S = {I}_{G} / {I}_{R}$$

where I_{G} and I_{R} are the fluorescence intensities of GQD and RQD, respectively.

For glycosylation analysis, the signal response is determined as below:

$$R = 1 - ( {S}_{s} / {S}_{o} )$$

where Sₒ and Sₛ are the fluorescence signals before and after sample incubation, respectively.

### Smartphone-Based Sensor

To enable smartphone-based evaluation of the STAR assay, we developed a sensor 500 consisting of five components (FIG. 24c): a 3D-printed optical cartridge 504, a UV LED source 506, a light diffuser 505, two optical filters 503, and a magnification lens 501. The optical cartridge was fabricated using a UV-curable resin (HTM 140) by a desktop 3D printer (EnvisionTEC, Aureus). The light source (Chaoziran S&T) was customized with a UV LED diode 506 with a central wavelength at 365 nm and the emitted UV light was spread homogeneously by a light diffuser 505 (Thorlabs DG05). Two bandpass filters 503 with center wavelengths of 520 nm and 610 nm were used for measuring fluorescence of GQD and RQD, respectively. The magnification lens 501 (Thorlabs LA4280) was set before the smartphone camera 501 to improve the image quality. The assembled system having dimensions 32 mm (width)×88 mm (length)×35 mm (height) was equipped with a sliding slot for quick attachment to the smartphone. The fluorescence images was processed by ImageJ software, to obtain the fluorescence intensity.

### Example 12. Identification of Glycan Signature in Clinical Samples

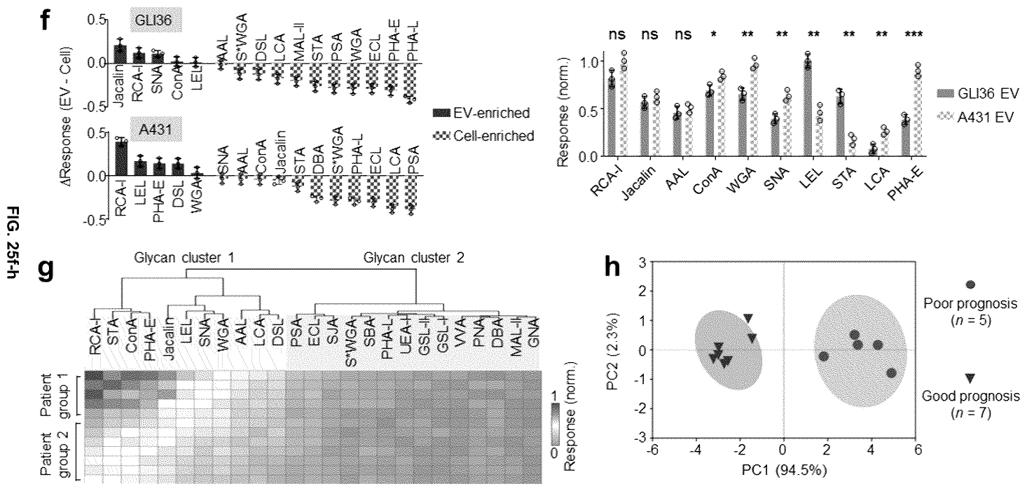

The dual-probe STAR developed in Example 11 was taken for the direct profiling of EV glycans in clinical biofluids. Specifically, the architecture contained two different types of QDs spatially organized within the assembly: RQD clustered centrally and GQD associated peripherally as dispersed entities. We grew the dual-probe STARs on a microfluidic platform and utilized their intra-assembly spatial distribution of nanoparticles (and hence different responsiveness) to develop the EV glycan assay (FIG. 25a), as described in Example 11. EVs were first incubated with the STARs to enable attachment through various interactions (e.g., electrostatic, zinc-carboxylate, and antigen-antibody, Cao, Y. et al., Biosens. Bioelectron. 2020, 166, 112452; and Gao, S. et al., Chem 2019, 5, 1597-1608). In the presence of EV glycan, the specific binding of lectin-oxidase generates H₂O₂ in situ: this liberation selectively quenches the fluorescence of the peripheral working probes (GQD), while leaving the central reference probes (RQD) unaffected (FIG. 25b). We thus determined the target glycan signatures by analyzing the relative changes in fluorescence intensities of the two probes.

### EV Isolation and Characterization

EVs derived from human brain glial cells (GLI36) and skin epithelial (A431) were collected through gradient centrifugation. Cells were cultured in DMEM supplemented with 5% dFBS, and penicillin-streptomycin. The culture medium was filtered through a 0.8-μm membrane filter (Millipore) and pelleted at 10,000 g for 20 min to deplete cell debris. The supernatant was centrifuged at 100,000 g for 2 h to concentrate EVs. Collected EVs were analyzed through nanoparticle tracking analysis (NTA) system (NS300, Nanosight) to quantify their size distribution and concentration. All NTA measurements were performed with identical system settings, with ˜ 50 vesicles in the field of view to achieve optimal counting. For TEM analysis of EVs, samples were fixed with 2% paraformaldehyde, loaded onto a copper grid (Latech), and contrast-stained with uranyl oxalate and methyl cellulose mixture before TEM analysis.

### ELISA Assay

Samples were adsorbed onto ELISA plates (Thermo Scientific) and blocked using PBS containing 1% BSA. After washing, biotinylated lectins (5 μg/ml) were introduced in PBS containing 1% BSA. Following incubation (1 h at room temperature), streptavidin-conjugated GQD-ZIF-8 probes were added. Fluorescence signal was determined through a commercial plate-reader (Tecan).

### Results and Discussion

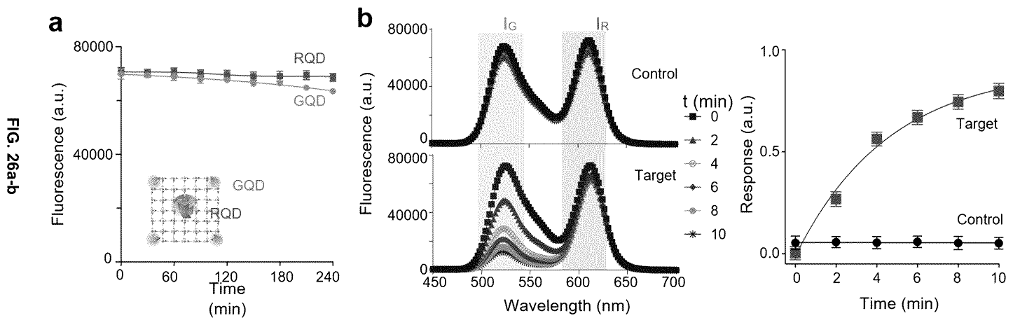

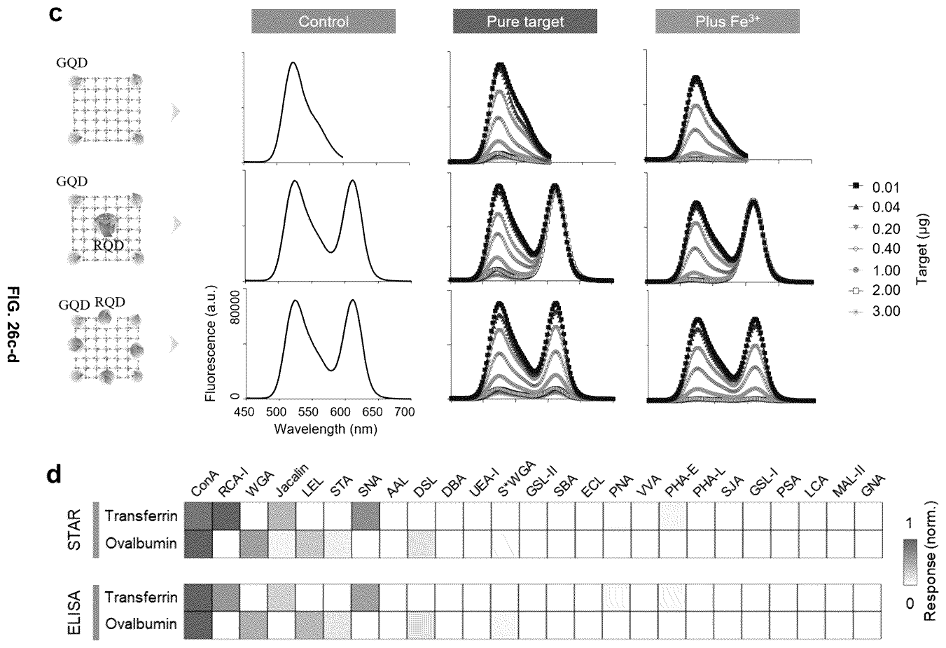

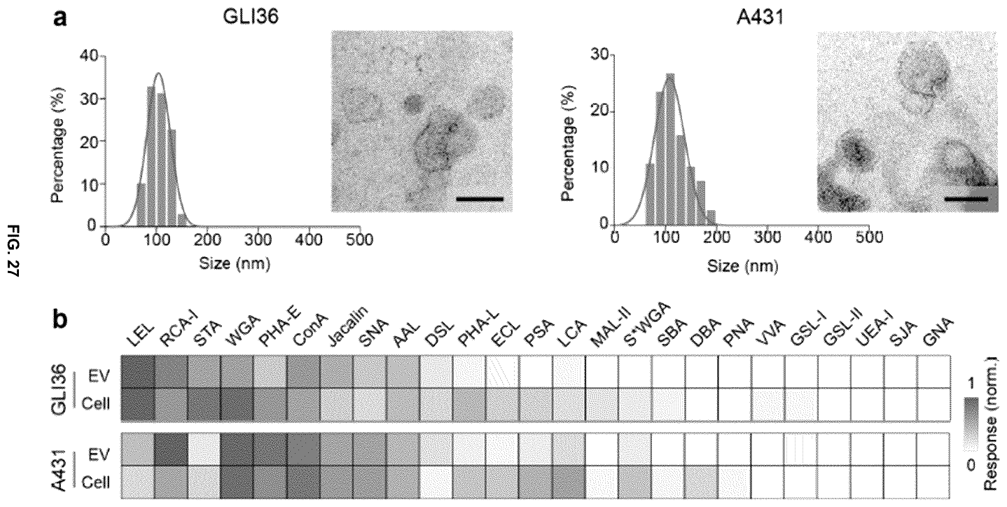

The STARs showed minimal nanoparticle leaching during the assay (FIG. 26a), and in the control experiment, demonstrated low fluorescence response in the absence of target glycan (FIG. 26b). As compared to other assemblies, the STAR's spatial probe arrangement enabled robust analysis, even in the presence of Fe³⁺ ions (Lou, Y. et al, J. Mater. Chem. C 2014, 2, 595-613), an interfering agent commonly found in clinical samples and known to quench QDs (FIGS. 25c and 26c). We further determined through a titration analysis that the STAR assay is >500-fold better than the gold-standard ELISA assay (FIG. 25d). Importantly, across different glycans measured (Table 2), the STAR analysis showed a good correlation (R²>92%) with the ELISA assay (FIGS. 25e and 26d). Employing the STAR assay, we next compared the glycan signatures of EVs and their parent cells. Specifically, we measured vesicles derived from brain glial cells (GLI36) and skin epithelial cells (A431) (FIG. 27). As compared to their parent cells, EVs were enriched with specific glycans (e.g., RCA-I, LEL). When evaluating vesicles derived from different cell origins, distinct glycan profiles could also be observed (FIG. 25f).

TABLE 2
List of lectins and their targeted glycan specificities.

| Abbreviation | Source/Name | Preferred glycan specificity |
|---|---|---|
| ConA | Concanavalin A | αMan, αGlc |
| SBA | Glycine max | α > βGalNAc |
| | (soybean) agglutinin | |
| WGA | Triticum vulgaris | GlcNAc, SA |
| | (wheat germ) | |
| | agglutinin | |
| DBA | Dolichos biflorus | αGalNAc |
| | agglutinin | |
| UEA-I | Ulex europaeus | (α-1,2) Fuc |
| | agglutinin I | |
| RCA120 | Ricinus communis | Gal |
| | agglutinin | |
| PNA | Arachis hypogaea | Galβ3GalNAc |
| | (peanut) agglutinin | |
| GSL-I | Griffonia (Bandeiraea) | αGal, αGalNAc |
| | simplicifolia lectin I | |
| PSA | Pisum sativum | αMan, αGlc |
| | agglutinin | |
| LCA | Lens culinaris | αMan, αGlc |
| | agglutinin | |
| PHA-E | Phaseolus vulgaris | Galβ4GlcNAcβ2Manα6(GlcNAcβ4) |
| | Erythroagglutinin | (GlcNAcβ4Manα3)Manβ4 |
| PHA-L | Phaseolus vulgaris | Galβ4GlcNAcβ6(GlcNAcβ2Manα3)Manα3 |
| | Leucoagglutinin | |
| SJA | Sophora japonica | βGalNAc |
| | (Japanese Pagoda Tree) | |
| SWGA | Wheat germ | GlcNAc |
| | agglutinin, | |
| | succinylated | |
| GSL-II | Griffonia (Bandeiraea) | α or βGlcNAc |
| | simplicifolia lectin II | |
| DSL | Datura Stramonium | (GlcNAc)2-4 |
| | lectin | |
| ECL | Erythrina cristagalli | Galβ4GlcNAc |
| | lectin | |
| Jacalin | Artocarpus integrifolia | Galβ3GalNAc |
| | (Jackfruit) | |
| LEL | Lycopersicon | (GlcNAc) 2-4 |
| | esculentum (tomato) | |
| | lectin | |
| STA | Solanum tuberosum | (GlcNAc) 2-4 |
| | (potatoe) lectin | |
| VVA | Vicia villosa agglutinin | GalNAc |
| SNA | Sambucus Nigra | (α-2,6) SA |
| | Lectin | |
| MAL-II | Maackia Amurensis | (α-2,3) SA |
| | Lectin II | |
| AAL | Aleuria aurantia lectin | (α-1,3) or (α-1,6) Fuc |
| GNA | Galanthus nivalis | (α-1,3) Man |
| | agglutinin | |

Glycan abbreviations:
Fuc: L-Fucose; Man: Mannose; GalNAc: N-Acetylgalactosamine; Gal: D-Galactose; SA: Sialic Acid; GlcNAc: N-Acetylglucosamine; Glc: D-Glucose

### Example 13. Clinical Utility of the STAR Assay

Finally, we conducted a feasibility study using colorectal cancer patient ascites. Using STARs functionalized with antibodies, we developed the assay to achieve direct glycan profiling of cancer-associated EVs in patient specimens, as described in Example 11. Specifically, we prepared STARs with antibodies against CD24 (Wang, Z. et al., Matter 2020, 2, 150-166; and Im, H. et al., Nat. Biotechnol. 2014, 32, 490-495), a known cancer antigen, to enrich and measure putative tumor-derived EVs.

### Clinical Measurements

The study was approved by the National University Hospital (2016/01088), and SingHealth (2015/2479) Institutional Review Boards. All subjects were recruited according to IRB-approved protocols after obtaining informed consent. Ascites samples were collected from colorectal cancer patients, centrifuged at 500 g for 10 min, and filtered through a 0.8-μm membrane filter (Millipore). All samples were de-identified and stored at −80° C. before glycan analysis.

Functionalisation of STAR Assemblies with Antibodies

The preparation of STAR assembly follows the protocol in Example 4. The STAR assembly was then incubated with antibody solution (2 μg/ml) for 1 h at room temperature to allow the immobilization of antibody molecules on the STAR assembly. After washing with PBS buffer, the STAR assembly was blocked with BSA solution (10 mg/ml) for further analysis.

For clinical analysis, ascites samples were used directly. To enable selective measurement of glycan signatures on EVs, we first functionalized STAR assemblies with antibodies against CD24, through electrostatic attraction and thiol-zinc affinity. Following antibody modification and subsequent BSA blocking, ascites samples (5 μl) were introduced for direct analysis, as described in Example 11. All STAR measurements were performed relative to respective sample-matched and no-lectin control. Clinical evaluation of patient characteristics was determined independently. Specifically, patient prognosis was determined by the overall survival from the time of collection of ascites. Patients were deemed to have a good prognosis when the overall survival was more than ten months. Conversely, patients were determined to have a poor prognosis if the overall survival was less than five months. All STAR measurements were performed blinded from these clinical evaluations.

### Results and Discussion

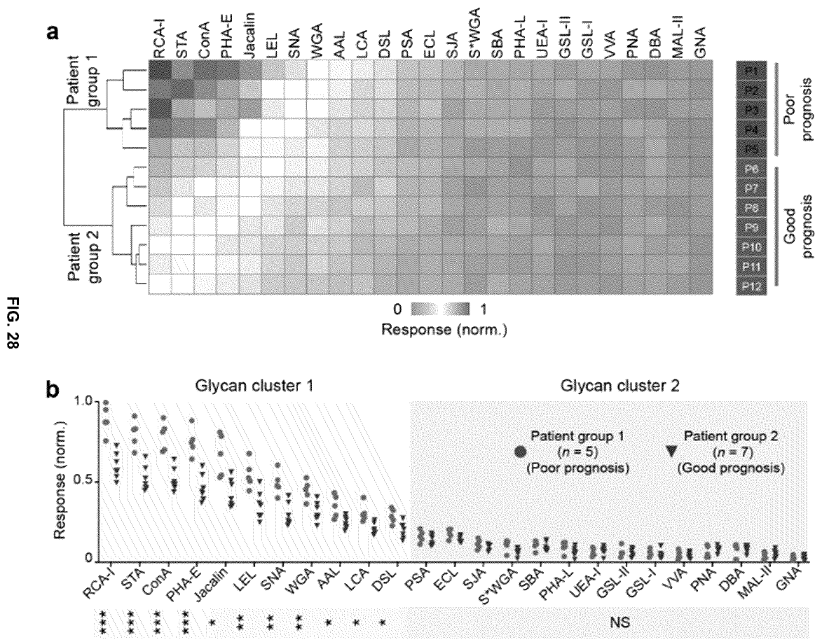

Multiplexed glycan profiling showed highly varied glycan signatures among the clinical samples (FIG. 25g). Hierarchical clustering of patient profiling data classified the patients into two populations and the glycan expressions into two clusters. This patient classification correlated well with independent clinical evaluation of prognosis based on patient survival data (FIG. 28a). We also found that glycan moieties of Cluster 1, but not those of Cluster 2, showed differential measurements across the two patient populations (FIG. 28b). Principal component analysis of Cluster 1 glycans enabled good differentiation of patient prognosis (FIG. 25h).

### General Discussion

The invention is able to provide a high degree of control over both the spatial control and integration versatility of nanoparticles/protein molecules within metal organic frameworks. This allows for a diverse range of nanotechnology applications as disclosed herein.

With respect to spatial control, the invention utilises surfactants to guide nanoparticle integration while moulding the growing framework. Unlike conventional approaches which either modify nanoparticles or template framework development, the invention leverages surfactant interactions to tune both simultaneously. Specifically, strong interactions between surfactants and metal organic framework constituents drive central integration and heterogeneous framework growth while weak interactions induce peripheral integration and homogeneous framework; likewise, strong interactions between surfactants and nanoparticles stabilize nanoparticles while weak interactions lead to clustered nanoparticles. The approach utilised by the invention is thus programmable and predictable from the general principles disclosed herein, and yields a high nanoparticle integration efficiency (>90%). It not only tunes the overall morphology but also achieves intra-assembly spatial control—nanoparticle organization and distribution—to develop hybrid architectures.

With respect to integration versatility, the approach can be readily expanded to assemble different nanomaterials with various hosts (e.g., 1D oriented, 3D epitaxial and amorphous metal organic frameworks). Importantly, unlike conventional synthesis approaches which require harsh chemicals and lengthy processing, the invention enables these composite metal organic framework to be prepared quickly and safely (<2 min aqueous synthesis at room temperature). The invention can therefore be adopted to integrate sensitive, biological molecules (e.g., proteins and small molecule drugs) and to achieve in situ growth on different types of solid substrates, to develop various multifunctional assemblies for diverse applications.

The invention also enables the design and development of metal organic frameworks comprising a number of different nanomaterials, each precisely positioned with respect to one another by rational surfactant selection.

1. A composite metal organic framework encapsulating one or more populations of particles, wherein a first population of particles comprise:
an outer shell region formed from a surfactant; and
an inner region comprising one or more of a nanoparticle and/or a plurality of protein molecules.

2. The composite metal organic framework according to claim 1, comprising a first population of particles and a second population of particles, where the second population of particles comprise:
an outer shell region formed from a surfactant; and
an inner region comprising one or more of a nanoparticle and/or a plurality of protein molecules, and
wherein the first population of particles are different to the second population of particles.

3. The composite metal organic framework according to claim 2, wherein:
the first population of particles are mono-dispersed within the metal organic framework; and
the second population of particles are present as particle aggregates within the metal organic framework.

4. The composite metal organic framework according to claim 3, wherein:
the interaction energy between the surfactant and the particles and/or protein molecules of the first population of particles is less than −83.7 kJ/mol (−20 kcal/mol), and
the interaction energy between the surfactant and the particles and/or protein molecules of the of the second population of particles is greater than −62.8 kJ/mol (−15 kcal/mol).

5. The composite metal organic framework of claim 2, wherein one or both of the following apply:
a majority of said first population of particles are located within a peripheral portion of the metal organic framework;
a majority of said second population of particles are located within a core portion of the metal organic framework.

6. The composite metal organic framework according to claim 5, wherein:
the interaction energy between the surfactant of the first population of particles and an organic linker material of the metal organic framework is greater than −41.8 kJ/mol (−10 kcal/mol), and
the interaction energy between the surfactant of the second population of particles and an organic linker material of the metal organic framework is less than −50.2 kJ/mol (−12 kcal/mol).

7. (canceled)

8. The composite metal organic framework according to claim 2, wherein the weight ratio of the first population of particles to the second population of particles ranges from 1:100 to 100:1.

9. The composite metal organic framework according to claim 1, wherein the first population of particles are located within a peripheral portion of the metal organic framework and wherein the interaction energy between the surfactant of the first population of particles and an organic linker material of the metal organic framework is greater than −41.8 kJ/mol (−10 kcal/mol).

10. (canceled)

11. The composite metal organic framework according to claim 1, wherein the first population of particles are located within a core portion of the metal organic framework and wherein the interaction energy between the surfactant of the first population of particles and an organic linker material of the metal organic framework is less than −50.2 kJ/mol (−12 kcal/mol).

12. (canceled)

13. The composite metal organic framework according to claim 1, wherein the first population of particles comprises particles mono-dispersed within the metal organic framework and wherein the interaction energy between the surfactant and the nanoparticles and/or protein molecules of the first population of particles is less than −83.7 kJ/mol (−20 kcal/mol).

14. (canceled)

15. The composite metal organic framework according to claim 1, wherein the first population of particles comprises particles present as particle aggregates within the metal organic framework and wherein the interaction energy between the surfactant and the particles and/or protein molecules of the first population of particles is greater than −62.8 kJ/mol (−15 kcal/mol).

16. (canceled)

17. The composite metal organic framework according to claim 2, wherein the first population of particles comprises a first set of quantum dots and wherein the second population of particles comprises a second set of quantum dots.

18. (canceled)

19. The composite metal organic framework according to claim 17, wherein said first set of quantum dots and second set of quantum dots are configured to emit light having different wavelengths.

20. (canceled)

21. (canceled)

22. (canceled)

23. (canceled)

24. (canceled)

25. A method of making a composite metal organic framework encapsulating a first population of particles, said method comprising the steps:
(a) providing a first population of particles, a metal node material and an organic linker material;
(b) reacting said first population of particles, metal node material and organic linker material in aqueous solution to form a composite metal organic framework where the first population of particles are entrapped by the metal organic framework,
wherein said metal node material and organic linker material are compatible to react together to form a metal organic framework, and
the first population of particles comprise:
an outer shell region formed from a surfactant; and
an inner region comprising one or more of a nanoparticle and/or a plurality of protein molecules.

26. (canceled)

27. (canceled)

28. (canceled)

29. (canceled)

30. (canceled)

31. (canceled)

32. (canceled)

33. (canceled)

34. The method according to claim 25, further comprising the preliminary steps:
(1) determining the desired distribution and dispersion of each population of particles within the resulting composite metal organic framework; and
(2) for each population of particles, selecting the surfactant, the nanoparticle or protein molecule, the metal node material and the organic linker material compatible to form the desired metal organic framework such that:
(A) if a majority of said population of particles is desired to exist as mono-dispersed particles, selecting the surfactant and the particle and/or protein molecule such that the interaction energy between the surfactant and the particles and/or protein molecules of said population of particles is less than −83.7 kJ/mol (−20 kcal/mol),
(B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is greater than −62.8 kJ/mol (−15 kcal/mol),
(C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is greater than −41.8 kJ/mol (−10 kcal/mol), and
(D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is less than −50.2 kJ/mol (−12 kcal/mol).

35. A method of selecting materials for forming a composite metal organic framework, said composite metal organic framework encapsulating one or more populations of particles, where each population of particles comprises:
an outer shell region formed from a surfactant; and
an inner region comprising one or more of a nanoparticle and/or a plurality of protein molecules,
said method comprising:
(1) determining the desired distribution and dispersion of each population of particles within the resulting composite metal organic framework; and
(2) for each population of particles, selecting the surfactant, the nanoparticle or protein molecule, and a metal node material and organic linker material compatible to form the desired metal organic framework such that:
(A) if a majority of said population of particles is desired to exist as mono-dispersed particles, selecting the surfactant and the particle and/or protein molecule such that the interaction energy between the surfactant and the particles and/or protein molecules of said population of particles is less than −83.7 kJ/mol (−20 kcal/mol),
(B) if a majority of said population of particles is desired to exist as particle aggregates, selecting the surfactant and the particles and/or protein molecules such that the interaction energy between the surfactant and the particles and/or protein molecules of said population of surfactant-coated nanoparticles is greater than −62.8 kJ/mol (−15 kcal/mol),
(C) if a majority of said population of particles is desired to be located in the periphery of the metal organic framework, selecting the surfactant, metal node material and organic linker material compatible to form the desired metal organic framework such that the interaction energy between the surfactant of said population of particles and the organic linker material of the metal organic framework is greater than −41.8 kJ/mol (−10 kcal/mol), and
(D) if a majority of said population of particles is desired to be located in the core of the metal organic framework, selecting the surfactant, metal node material and organic linker material compatible to form the desired metal organic framework such that the interaction energy between the surfactant of said population of particles and the organic linker material of the metal organic framework is less than −50.2 kJ/mol (−12 kcal/mol).

36. (canceled)

37. (canceled)

38. A diagnostic method comprising:
(a) contacting a composite metal organic framework as defined in claim 18 with an isolated biological sample;
(b) measuring the photoluminescence of the first and second set of quantum dots;
(c) determining the presence or absence of a biological marker indicated in a disease state by comparing the photoluminescence of the first and second set of quantum dots with each other or with a reference value, where said biological marker, if present, is able to selectively quench fluorescence of the first or second set of quantum dots; and
(d) assigning the presence or absence of a disease state based on the presence or absence of said biological marker.

39. A composite metal organic framework according to claim 1, wherein the nanoparticle is doxorubicin and/or the protein molecule is bovine serum albumin (BSA).

40. A composite product comprising:
a substrate; and
a composite metal organic framework according to claim 1, wherein the composite metal organic framework is coated over the whole or part of the surface of said substrate.

41. (canceled)

42. A pharmaceutical composition comprising a composite metal organic framework according to claim 1, wherein the inner region of the first population of particles comprises:
a nanoparticle comprising a small molecule active agent; and/or
a plurality of protein molecules.
